(19) United States
(12) Patent Application Publication
(10) Pub. No.: US 2022/0036968 A1
(43) Pub. Date: Feb. 3, 2022

(54) PROCESSING BIOPHYSICAL SCREENING DATA AND IDENTIFYING AND CHARACTERIZING PROTEIN SITES FOR DRUG DISCOVERY

(71) Applicant: Frontier Medicines Corporation, South San Francisco, CA (US)

(72) Inventors: Saulo DE OLIVEIRA, San Francisco, CA (US); Johannes Hermann, San Francisco, CA (US); Chris Varma, Portola Valley, CA (US)

(73) Assignee: Frontier Medicines Corporation, South San Francisco, CA (US)

(21) Appl. No.: 17/444,018

(22) Filed: Jul. 29, 2021

Related U.S. Application Data
(60) Provisional application No. 63/059,096, filed on Jul. 30, 2020.
(60) Provisional application No. 63/059,099, filed on Jul. 30, 2020.
(60) Provisional application No. 63/059,100, filed on Jul. 30, 2020.

(51) Int. Cl. G16B 15/30; G16B 40/00; G16B 50/30; G16B 45/00

(52) U.S. Cl. CPC G16B 15/30; G16B 45/00; G16B 50/30; G16B 40/00

(57) ABSTRACT

Techniques for characterizing protein candidate sites are provided. Experimental data comprising spectral data from an experimental data source is received. Based on the experimental data, a data set comprising a set of protein candidate sites within one or more proteins is created. For each protein candidate site of the set of protein candidate sites, a feature set characterizing the respective protein candidate site is generated. A characterization of the amenability for drug-discovery for one or more of the protein candidate sites is generated by applying a classifier to the respective feature set for the protein candidate site.

### CROSS-REFERENCE TO RELATED APPLICATIONS

This application claims benefit of U.S. Provisional Patent Application No. 63/059,096, filed Jul. 30, 2020; U.S. Provisional Patent Application No. 63/059,099, filed Jul. 30, 2020; and U.S. Provisional Patent Application No. 63/059,100, filed Jul. 30, 2020, the entire contents of each of which are hereby incorporated by reference.

### FIELD OF THE INVENTION

This relates generally to systems and methods for automated database management and automated analysis of reactive sites on proteins, and more specifically to systems and methods for automated ingestion of data from biophysical screening experiments for the creation of a database storing candidate sites and for analyzing protein reactive sites to assess reactivity and/or amenability for drug discovery.

### BACKGROUND

A majority of drugs exert their therapeutic effect by interacting with a protein or proteins involved in a disease pathway. Such proteins can be targeted for screening with candidate drugs, to determine the ability of the candidate drug to alter the activity of the protein. Proteins are said to be “druggable” when a therapeutic agent, such as a small molecule, interacts with the protein with sufficient affinity to alter the activity of the protein.

In order for a drug to be effective against a protein, it needs to first bind to it. Approximately 90% of human proteins have no known binding site on the surface of the protein, complicating the discovery and development of drugs against those protein targets. However, chemoproteomics technology can be used to identify reactive sites in the human proteome. This enables previously undruggable proteins to be accessible to drug discovery and development for therapeutic intervention.

Chemoproteomics involves the study of a proteome by chemical and biophysical methods. One useful chemoproteomic tool is activity-based protein profiling (ABPP), where a probe is used to explore the reactivity of proteins. The ABPP probe typically consists of a reactive functionality, or “warhead,” that reacts irreversibly with a protein; a linker group, which may also bias the probe towards binding and reacting with specific proteins; and a reporter group or tag for identification of the probe-protein complex. For example, N-5-hexyn-1-yl-2-iodoacetamide has been used to explore reactivity of cysteine residues, which react with the iodoacetamide group, while the alkynyl group can be derivatized with further labels such as biotin or isotopically labeled moieties in order to tag the probe-protein complex (see, e.g., Weerapana et al. (2010), Nature 468(7325): 790-795).

A wide variety of chemoproteomic experiments are available, which can identify reactive proteins and reveal reactive sites in proteins. Many of the methods can be used in complex mixtures of proteins, such as entire proteomes. Other methods can be used to study isolated proteins in more detail. Chemoproteomics can thus generate large amounts of information about large numbers of proteins under a wide variety of conditions. A typical proteome-wide chemoproteomics experiment can generate up to tens of thousands of peptides and sites spanning thousands of proteins. Considering that multiple conditions are often tested and compared, and the fact that experiments are carried out in replicates, data for millions of peptides and sites can be produced in a short period of time such as within a month, a period which will shorten as experimental technology advances.

Managing the massive amount of information that chemoproteomics provides poses a formidable challenge. Experiments can be run on different samples, using different probes, under different reaction conditions. These experimental variables can affect the quality of the information derived. Also, interpretation of the experiments often depends on input from other data sources, such as protein sequence databases, which are continually updated, expanded, and corrected. Thus, for example, chemoproteomics data which depended on sequence information available at a particular point in time may need to be re-analyzed when the sequence information is updated.

The present disclosure provides databases and data management methods for recording, analyzing, and updating experimental chemoproteomic data, which permits judicious evaluation and assessment of the data, and interpretations of the data for use in fields such as drug discovery and drug development.

The data compiled from chemoproteomics experiments is useful in its raw form, but in many cases does not permit prioritization between candidate sites in different proteins, or between different druggable sites within the same protein. Screening strategies which do not correct for biases in the data, or which do not use machine learning, are risky and less efficient, since incorrectly perceived interesting sites could be pursued and favorable sites could be disregarded. This leads to higher drug discovery costs, or even failure in drug discovery, since sites that were not recognized as promising and not investigated could be ignored.

The present disclosure provides data processing methods for ranking or scoring protein reactive sites, either by using analytical techniques or by using machine learning, in order to assess the utility of such reactive sites for drug discovery, drug development, and other related uses.

### SUMMARY

The present disclosure provides systems, databases, and methods for input of experimental data involving chemoproteomics experiments, such as protein modification experiments and/or protein labeling experiments; analysis of the data; and generation of a database of candidate sites in the protein for use in drug discovery and development. In some embodiments, the system comprises a data ingestion engine which automatically ingests experimental data, annotated with the experimental conditions under which the data was generated. In some embodiments, the data ingestion engine uses a comprehensive set of defined fields encompassing all possible experimental conditions under which chemoproteomics screening is performed in order to process the experimental data consistently, but is flexible in terms of which descriptions it accepts and provides for facile expansion of the description of experimental conditions and the set of defined fields. In some embodiments, the data ingestion engine then processes the experimental data to generate a database of candidate sites in proteins, drawing on internal and external data sources to correlate experimental data with structural and sequence data in the proteins. In some embodiments, changes in information in the data sources are monitored, such as additions or corrections to protein databases, or additional or corrected experimental data, and the data is re-processed as appropriate to use the most accurate information available. Prior chemoproteomics work has used varying experimental conditions, which are often inconsistent and which may not be directly comparable, and the results are typically presented as a static data set, without the capability of updating if new information is added to a database or if corrections are made to previous information on a database. The lower reliability of the identification of candidate sites identified in proteins in these prior methods may lead to wasted effort.

As an example of processing which can be carried out by system such as those disclosed herein, a chemoproteomics experiment can be performed which involves incubating a protein, or a mixture of proteins, with a probe that specifically reacts with cysteine amino acids. Following the incubation, the protein or proteins may be digested with a protease such as trypsin. The peptide fragments resulting from digestion may then be analyzed by a mass spectrometric method such as LC/tandem mass spectrometry, resulting in a collection of experimental mass spectra.

In some embodiments, the data ingestion engine can retrieve protein sequences from a data source, such as UniProt or GenBank. In some embodiments, theoretical mass spectrometry data is generated from the database sequences; for the example above, the data ingestion engine may identify peptide fragments of the protein sequences, including peptide fragments of the proteins that would be produced by trypsin digestion, and calculate the theoretical mass spectra resulting from those fragments. In some embodiments, since the molecular weight of the probe is known, the theoretical mass spectra can include both peptides that have been modified with the probe as well as unmodified peptides. In some embodiments, a comparison of the experimental mass spectra with the theoretical mass spectra permits identification of the peptide observed in the experiment, and of the candidate site in the peptide. For example, if masses (M/Z) of 1,100 and 1,179 are observed in the experimental mass spectra, and if there is only one peptide fragment in the theoretical mass spectra generated for the proteins retrieved from the data source with a mass of 1,100, the experimental data can be mapped to that region of the protein. Further, if it is known that the probe adds 79 Daltons to a peptide when it reacts with cysteine, and the region of the protein identified with the mass of 1,100 contains a cysteine amino acid, then the cysteine amino acid in that region of the protein can, in some embodiments, be identified as the location where the probe bound to the peptide fragment, and registered as a candidate site in the database. In some embodiments, various factors, such as experimental error (e.g., the difference between a measured value and the true value) or noise, may prevent such an analysis from reaching 100% accuracy. In some embodiments, statistical analysis can be used to account for noise and experimental error before the candidate site is registered in the database. In some embodiments, a confidence value for the mapping of the experimental spectrum to the theoretical spectrum can be assigned for each spectral mapping.

The present disclosure provides methods for ranking protein reactive sites, in order to accurately identify and prioritize protein reactive sites suitable for drug discovery, drug development, or other uses. In some embodiments, the methods can be applied to a database of chemoproteomic information containing data compiled about the reactive sites. Application of the methods to a database of chemoproteomics information can, in some embodiments, provide rankings of protein sites which indicate the usefulness of the sites for their intended purpose.

In some embodiments, such rankings can take into account factors beyond the chemoproteomic data, in order to improve ranking and incorporate other desirable properties of protein reactive sites beyond what can be captured by chemoproteomics alone. An example is normalizing for protein concentration in a sample. In some embodiments, normalizing for concentration can help distinguish a protein reactive site that is detected frequently in chemoproteomics experiments because of high reactivity, and thus may be a promising candidate site, from a protein reactive site that is detected frequently simply because the protein is present at higher concentration, and which may not be a promising candidate (although other factors can also be weighed in ranking the sites). Additional procedures are disclosed herein for ranking, which, in some embodiments, can identify protein reactive sites suitable for a desired use such as drug discovery and drug development.

In some embodiments, a first system, for characterizing protein candidate sites, is provided, the first system comprising one or more processors configured to cause the system to: receive experimental data comprising spectral data from an experimental data source; in response to receiving the experimental data comprising the spectral data, automatically create, based on the experimental data, a data set comprising a set of protein candidate sites within one or more proteins; for each protein candidate site of the set of protein candidate sites, generate, based on the data set comprising the set of protein candidate sites, a feature set characterizing the respective protein candidate site; and generate a characterization of the amenability for drug-discovery for one or more of the protein candidate sites by applying a classifier to the respective feature set for the protein candidate site.

In some embodiments of the first system, the one or more processors are further configured to cause the first system to automatically generate, based on the received experimental data comprising the spectral data, for each peptide of a plurality of peptides, respective mapping data that maps a respective one of the peptides to a respective plurality of proteins; automatically creating the data set comprising the set of protein candidate sites is performed based on the respective pluralities of proteins indicated by the generated mapping data; and the one or more proteins are within in the respective pluralities of proteins.

In some embodiments of the first system, automatically creating the data set comprising the set of protein candidate sites comprises retrieving, from a protein sequence data source, protein annotation information indicating that the protein candidate sites are associated with one or more of the one or more proteins.

In some embodiments of the first system, the one or more processors are further configured to cause the first system to: detect an update to the protein sequence data source; and in response to detecting the update to the protein sequence data source, automatically update the data set comprising the set of protein candidate sites based on updated information retrieved from the updated protein sequence data source.

In some embodiments of the first system, automatically updating the data set comprising the set of protein candidate sites comprises performing one or more sequence alignments for a peptide of the plurality of peptides.

In some embodiments of the first system, performing the one or more sequence alignments for the peptide comprises aligning the peptide against updated protein sequence information for each of the proteins to which the peptide was previously indicated, by the mapping data, as having been mapped.

In some embodiments of the first system, automatically updating the data set comprising the set of protein candidate sites comprises aligning each peptide of the plurality of peptides against a new protein sequence added to the protein sequence data store.

In some embodiments of the first system, the updated information retrieved from the updated protein sequence data store comprises one or more of: information indicating a single-residue change, information indicating an insertion of an amino acid, information indicating a deletion of an amino acid, information indicating a novel protein annotation, information indicating merging of two or more protein entries into a single protein entry, and information indicating deletion of a protein entry.

In some embodiments of the first system, the plurality of proteins comprises one or more of a protein isoform and a protein mutant.

In some embodiments of the first system, the one or more processors are further configured to cause the first system to store the data set comprising the set of protein candidate sites in a database.

In some embodiments of the first system, the one or more processors are further configured to cause the first system to store the generated mapping data in a database.

In some embodiments of the first system, generating the mapping data comprises correlating spectra from within the received spectral data to matching spectra from calculated theoretical spectra.

In some embodiments of the first system, the one or more processors are further configured to cause the first system to calculate the theoretical spectra based on protein sequence data received by the system from a protein sequence data source.

In some embodiments of the first system, the one or more processors are further configured to cause the first system to automatically generate and store in a database a sequence of one or more peptides comprising the protein candidate sites.

In some embodiments of the first system, the one or more processors are further configured to cause the first system to generate a score characterizing a confidence level associated with at least part of the data set comprising the set of protein candidate sites.

In some embodiments of the first system, the one or more processors are further configured to cause the first system to, in response to receiving the experimental data comprising the spectral data, store the experimental data comprising the spectral data in a database.

In some embodiments of the first system, the one or more processors are further configured to cause the first system to: receive metadata specifying experimental conditions for the experimental data source, wherein the data is received via a plurality of predefined fields for experimental condition data types; and in response to receiving the metadata specifying the experimental conditions, generate and store a record of the experimental conditions in a database.

In some embodiments of the first system, the spectral data comprises mass spectrometry data.

In some embodiments of the first system, the spectral data comprises tandem mass spectrometry data.

In some embodiments of the first system, the spectral data is received by the system before being associated with any peptides or proteins.

In some embodiments of the first system, the data set comprising the set of protein candidate sites comprises indication of a set of candidate residues determined by the system to be potentially modified by promiscuous probes.

In some embodiments of the first system, the experimental data is data generated by one or more of a cell-based screening experiment, an in vitro screening experiment, an in situ screening experiment, an in vivo screening experiment, a purified protein screening experiment, and a recombinant protein screening experiment.

In some embodiments of the first system, the feature set comprises: one or more features characterizing frequency of observation of one or more peptides associated with the respective protein candidate site across one or more experimental iterations, as indicated by metadata corresponding to the respective protein candidate site; one or more features characterizing protein abundance for a respective protein comprising the respective protein candidate site; and one or more features characterizing sequence characteristics associated with the respective protein candidate site.

In some embodiments of the first system, the one or more features characterizing frequency of observation of the one or more peptides associated with the respective protein candidate site comprise a first feature characterizing a number of times that the one or more peptides associated with the respective protein candidate site were observed across the one or more experimental iterations.

In some embodiments of the first system, the first feature characterizes a number of times that one peptide associated with the respective protein candidate site was observed across the one or more experimental iterations.

In some embodiments of the first system, the one or more features characterizing frequency of observation of the one or more peptides associated with the respective protein candidate site comprise a second feature characterizing a number of experimental iterations in which the one or more peptides associated with the respective protein candidate site were observed.

In some embodiments of the first system, the second feature characterizes a number of experimental iterations in which one peptide associated with the respective protein candidate site was observed.

In some embodiments of the first system, the one or more features characterizing frequency of observation of the one or more peptides associated with the respective protein candidate site comprise a third feature characterizing a percentage of experimental observations of the one or more peptides associated with the respective protein candidate site in which the one or more peptides are observed as modified.

In some embodiments of the first system, the third feature characterizes a percentage of experimental observations of one peptide associated with the respective protein candidate site in which the one peptide is observed as modified.

In some embodiments of the first system, the one or more features characterizing frequency of observation of the one or more peptides associated with the respective protein candidate site comprise a fourth feature characterizing a percentage of experiments in which the one or more peptides associated with the respective protein candidate site are observed in which the one or more peptides are observed as modified.

In some embodiments of the first system, the fourth feature characterizes a percentage of experiments in which one peptide associated with the respective protein candidate site is observed in which the one peptide is observed as modified.

In some embodiments of the first system, the one or more features characterizing protein abundance comprise a fifth feature characterizing protein abundance data retrieved from a protein abundance data source.

In some embodiments of the first system, the one or more features characterizing sequence characteristics comprise a sixth feature characterizing a number of charged residues associated with the respective protein candidate site.

In some embodiments of the first system, the feature set comprises one or more features characterizing additional aspects of experimental observation, distinct from the one or more features characterizing frequency of observation, of one or more peptides associated with the respective protein candidate site.

In some embodiments of the first system, the one or more features characterizing additional aspects comprise a seventh feature characterizing a number of experimental iterations, indicated by the metadata corresponding to the respective protein candidate site, that include one or more peptides in a modified or unmodified state.

In some embodiments of the first system, the characterization of the amenability for drug-discovery of the protein candidate site comprises a probability of the protein candidate site being reactive.

In some embodiments of the first system, the one or more processors are further configured to cause the first system to generate and store a ranking of the set of protein candidate sites, wherein the ranking is based on the characterization of the amenability for drug-discovery for one or more of the protein candidate sites generated by the classifier.

In some embodiments, a first method, for characterizing protein candidate sites, is provided, the first method performed at a system comprising one or more processors, the method comprising: receiving experimental data comprising spectral data from an experimental data source; in response to receiving the experimental data comprising the spectral data, automatically creating, based on the experimental data, a data set comprising a set of protein candidate sites within one or more proteins; for each protein candidate site of the set of protein candidate sites, generating, based on the data set comprising the set of protein candidate sites, a feature set characterizing the respective protein candidate site; and generating a characterization of the amenability for drug-discovery for one or more of the protein candidate sites by applying a classifier to the respective feature set for the protein candidate site.

In some embodiments, a first non-transitory computer-readable storage medium, for characterizing protein candidate sites, is provided, the first non-transitory computer-readable storage medium storing instructions configured to be executed by a system comprising one or more processors to cause the system to: receive experimental data comprising spectral data from an experimental data source; in response to receiving the experimental data comprising the spectral data, automatically create, based on the experimental data, a data set comprising a set of protein candidate sites within one or more proteins; for each protein candidate site of the set of protein candidate sites, generate, based on the data set comprising the set of protein candidate sites, a feature set characterizing the respective protein candidate site; and generate a characterization of the amenability for drug-discovery for one or more of the protein candidate sites by applying a classifier to the respective feature set for the protein candidate site.

In some embodiments, a second system, for ingesting data from biophysical screening experiments, is provided, the second system comprising one or more processors configured to cause the second system to: receive experimental data comprising spectral data from an experimental data source; in response to receiving the experimental data comprising the spectral data, automatically generate, based on the received experimental data comprising the spectral data, for each peptide of a plurality of peptides, respective mapping data that maps a respective one of the peptides to one or more proteins of a respective plurality of proteins; automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and store the data set comprising the set of protein candidate sites in a first database.

In some embodiments of the second system, the one or more processors are further configured to cause the second system to store the generated mapping data in the first database.

In some embodiments of the second system, generating the mapping data comprises correlating spectra from within the received spectral data to matching spectra from calculated theoretical spectra.

In some embodiments of the second system, the one or more processors are further configured to cause the second system to calculate the theoretical spectra based on protein sequence data received by the system from a protein sequence data source.

In some embodiments of the second system, the one or more processors are further configured to cause the second system to automatically generate and store in the first database a sequence of one or more peptides comprising the protein candidate sites.

In some embodiments of the second system, the one or more processors are further configured to cause the second system to generate a score characterizing a confidence level associated with at least part of the data set comprising the set of protein candidate sites.

In some embodiments of the second system, the one or more processors are further configured to cause the second system to: detect an update to the protein sequence data source; and in response to detecting the update to the protein sequence data source, automatically update the data set comprising the set of protein candidate sites based on updated information retrieved from the updated protein sequence data source.

In some embodiments of the second system, automatically updating the data set comprising the set of protein candidate sites comprises performing one or more sequence alignments for a peptide of the plurality of peptides.

In some embodiments of the second system, performing the one or more sequence alignments for the peptide comprises aligning the peptide against updated protein sequence information for each of the proteins to which the peptide was previously indicated, by the mapping data, as having been mapped.

In some embodiments of the second system, automatically updating the data set comprising the set of protein candidate sites comprises aligning each peptide of the plurality of peptides against a new protein sequence added to the protein sequence data store.

In some embodiments of the second system, the updated information retrieved from the updated protein sequence data store comprises one or more of: information indicating a single-residue change, information indicating an insertion of an amino acid, information indicating a deletion of an amino acid, information indicating a novel protein annotation, information indicating merging of two or more protein entries into a single protein entry, and information indicating deletion of a protein entry.

In some embodiments of the second system, the one or more processors are further configured to cause the second system to, in response to receiving the experimental data comprising the spectral data, store the experimental data comprising the spectral data in a second database.

In some embodiments of the second system, the one or more processors are further configured to cause the second system to: receive metadata specifying experimental conditions for the experimental data source, wherein the metadata is received via a plurality of predefined fields for experimental condition data types; and in response to receiving the metadata specifying the experimental conditions, generate and store a record of the experimental conditions in the first database.

In some embodiments of the second system, the spectral data comprises mass spectrometry data.

In some embodiments of the second system, the spectral data comprises tandem mass spectrometry data.

In some embodiments of the second system, the spectral data is received by the system before being associated with any peptides or proteins.

In some embodiments of the second system, the data set comprising the set of protein candidate sites comprises indication of a set of candidate residues determined by the system to be potentially modified by promiscuous probes.

In some embodiments of the second system, the experimental data is data generated by one or more of a cell-based screening experiment, an in vitro screening experiment, an in situ screening experiment, an in vivo screening experiment, a purified protein screening experiment, and a recombinant protein screening experiment.

In some embodiments of the second system, the plurality of proteins comprises one or more of a protein isoform and a protein mutant.

In some embodiments, a second method, for ingesting data from biophysical screening experiments, is provided, the second method performed at a system comprising one or more processors, the second method comprising: receiving experimental data comprising spectral data from an experimental data source; in response to receiving the experimental data comprising the spectral data, automatically generating, based on the received experimental data comprising the spectral data, for each peptide of a plurality of peptides, respective mapping data that maps a respective one of the peptides to one or more proteins of a respective plurality of proteins; automatically creating, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and storing the data set comprising the set of protein candidate sites in a first database.

In some embodiments, a second non-transitory computer-readable storage medium, for ingesting data from biophysical screening experiments, is provided, the second non-transitory computer-readable storage medium storing instructions configured to be executed by a system comprising one or more processors to cause the system to: receive experimental data comprising spectral data from an experimental data source; in response to receiving the experimental data comprising the spectral data, automatically generate, based on the received experimental data comprising the spectral data, for each peptide of a plurality of peptides, respective mapping data that maps a respective one of the peptides to one or more proteins of a respective plurality of proteins; automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and store the data set comprising the set of protein candidate sites in a first database.

In some embodiments, a third system, for characterizing protein candidate sites, is provided, the third system comprising one or more processors configured to cause the third system to: receive data comprising a set of protein candidate sites and corresponding metadata regarding experimental data associated with respective protein candidate sites of the set of protein candidate sites; for each protein candidate site of the set of protein candidate sites, generate, based on the received data, a feature set characterizing the respective protein candidate site, wherein the feature set comprises: one or more features characterizing frequency of observation of one or more peptides associated with the respective protein candidate site across one or more experimental iterations, as indicated by metadata corresponding to the respective protein candidate site; one or more features characterizing protein abundance for a respective protein comprising the respective protein candidate site; and one or more features characterizing sequence characteristics associated with the respective protein candidate site; and generate a characterization of the amenability for drug-discovery for one or more of the protein candidate sites by applying a classifier to the respective feature set for the protein candidate site.

In some embodiments of the third system, the characterization of the amenability for drug-discovery of the protein candidate site comprises a probability of the protein candidate site being reactive.

In some embodiments of the third system, the one or more processors are further configured to cause the third system to generate and store a ranking of the set of protein candidate sites, wherein the ranking is based on the characterization of the amenability for drug-discovery for one or more of the protein candidate sites generated by the classifier.

In some embodiments of the third system, the one or more features characterizing frequency of observation of the one or more peptides associated with the respective protein candidate site comprise a first feature characterizing a number of times that the one or more peptides associated with the respective protein candidate site were observed across the one or more experimental iterations.

In some embodiments of the third system, the first feature characterizes a number of times that one peptide associated with the respective protein candidate site was observed across the one or more experimental iterations.

In some embodiments of the third system, the one or more features characterizing frequency of observation of the one or more peptides associated with the respective protein candidate site comprise a second feature characterizing a number of experimental iterations in which the one or more peptides associated with the respective protein candidate site were observed.

In some embodiments of the third system, the second feature characterizes a number of experimental iterations in which one peptide associated with the respective protein candidate site was observed.

In some embodiments of the third system, the one or more features characterizing frequency of observation of the one or more peptides associated with the respective protein candidate site comprise a third feature characterizing a percentage of experimental observations of the one or more peptides associated with the respective protein candidate site in which the one or more peptides are observed as modified.

In some embodiments of the third system, the third feature characterizes a percentage of experimental observations of one peptide associated with the respective protein candidate site in which the one peptide is observed as modified.

In some embodiments of the third system, the one or more features characterizing frequency of observation of the one or more peptides associated with the respective protein candidate site comprise a fourth feature characterizing a percentage of experiments in which the one or more peptides associated with the respective protein candidate site are observed in which the one or more peptides are observed as modified.

In some embodiments of the third system, the fourth feature characterizes a percentage of experiments in which one peptide associated with the respective protein candidate site is observed in which the one peptide is observed as modified.

In some embodiments of the third system, the one or more features characterizing protein abundance comprise a fifth feature characterizing protein abundance data retrieved from a protein abundance data source.

In some embodiments of the third system, the one or more features characterizing sequence characteristics comprise a sixth feature characterizing a number of charged residues associated with the respective protein candidate site.

In some embodiments of the third system, the feature set comprises one or more features characterizing additional aspects of experimental observation, distinct from the one or more features characterizing frequency of observation, of one or more peptides associated with the respective protein candidate site.

In some embodiments of the third system, the one or more features characterizing additional aspects comprise a seventh feature characterizing a number of experimental iterations, indicated by the metadata corresponding to the respective protein candidate site, that include one or more peptides in a modified or unmodified state.

In some embodiments, a third method, for characterizing protein candidate sites, is provided, the third method performed at a system comprising one or more processors, the third method comprising: receiving data comprising a set of protein candidate sites and corresponding metadata regarding experimental data associated with respective protein candidate sites of the set of protein candidate sites; for each protein candidate site of the set of protein candidate sites, generating, based on the received data, a feature set characterizing the respective protein candidate site, wherein the feature set comprises: one or more features characterizing frequency of observation of one or more peptides associated with the respective protein candidate site across one or more experimental iterations, as indicated by metadata corresponding to the respective protein candidate site; one or more features characterizing protein abundance for a respective protein comprising the respective protein candidate site; and one or more features characterizing sequence characteristics associated with the respective protein candidate site; and generating a characterization of the amenability for drug-discovery for one or more of the protein candidate sites by applying a classifier to the respective feature set for the protein candidate site.

In some embodiments, a third non-transitory computer-readable storage medium, for characterizing protein candidate sites, is provided, the third non-transitory computer-readable storage medium storing instructions configured to be executed by a system comprising one or more processors to cause the system to: receive data comprising a set of protein candidate sites and corresponding metadata regarding experimental data associated with respective protein candidate sites of the set of protein candidate sites; for each protein candidate site of the set of protein candidate sites, generate, based on the received data, a feature set characterizing the respective protein candidate site, wherein the feature set comprises: one or more features characterizing frequency of observation of one or more peptides associated with the respective protein candidate site across one or more experimental iterations, as indicated by metadata corresponding to the respective protein candidate site; one or more features characterizing protein abundance for a respective protein comprising the respective protein candidate site; and one or more features characterizing sequence characteristics associated with the respective protein candidate site; and generate a characterization of the amenability for drug-discovery for one or more of the protein candidate sites by applying a classifier to the respective feature set for the protein candidate site.

In some embodiments, a fourth system, for training a classifier for identifying protein candidate sites, is provided, the fourth system comprising one or more processors configured to cause the fourth system to: receive a corpus of training data comprising data regarding a plurality of protein candidate sites; generate, based on the training data, a plurality of feature sets corresponding to the plurality of protein candidate sites; and train a classifier using the plurality of feature sets to classify protein candidate sites for amenability for drug-discovery.

In some embodiments of the fourth system, the training data set comprises cysteine residue information from a protein sequence data source.

In some embodiments of the fourth system, receiving the training data set comprises selecting the plurality of protein candidate sites from a protein sequence data source in accordance with metadata indicating one of (a) that the protein candidate sites are catalytic and (b) that the protein sites are not catalytic.

In some embodiments of the fourth system, receiving the training data set comprises selecting the plurality of protein candidate sites from a protein sequence data source in accordance with metadata indicating that the protein candidate sites are post-translationally modified.

In some embodiments of the fourth system, receiving the training data set comprises selecting the plurality of protein candidate sites from a protein sequence data source in accordance with metadata indicating that the protein candidate sites are amenable for drug discovery.

In some embodiments of the fourth system, receiving the training data set comprises selecting the plurality of protein candidate sites from a protein sequence data source in accordance with metadata indicating that the protein candidate sites are associated with a numerical score for known drug discovery amenability satisfying one or more predefined threshold criteria.

In some embodiments of the fourth system, receiving the training data set comprises selecting the plurality of protein candidate sites from a protein sequence data source in accordance with metadata indicating that the protein candidate sites satisfy one or more protein abundance criteria.

In some embodiments of the fourth system, receiving the training data set comprises selecting the plurality of protein candidate sites from a protein sequence data source in accordance with metadata indicating that the protein candidate sites satisfy one or more isotopic ratio criteria.

In some embodiments of the fourth system, receiving the training data set comprises selecting the plurality of protein candidate sites from a protein sequence data source in accordance with metadata indicating that the protein candidate sites satisfy one or more reactivity criteria.

In some embodiments of the fourth system, training the classifier comprises applying a model selected from Support Vector Machines (SVM), Random Forests (RF), and eXtreme Gradient Boosting (XGBoost).

In some embodiments of the fourth system, each of the feature sets of the plurality of feature sets comprises: one or more features characterizing frequency of observation of one or more peptides associated with the respective protein candidate site across one or more experimental iterations, as indicated by metadata corresponding to the respective protein candidate site; one or more features characterizing protein abundance for the respective protein candidate site; and one or more features characterizing sequence characteristics associated with the respective protein candidate site.

In some embodiments, a fourth method, for training a classifier for identifying protein candidate sites, is provided, the fourth method performed at a system comprising one or more processors, the fourth method comprising: receiving a corpus of training data comprising data regarding a plurality of protein candidate sites; generating, based on the training data, a plurality of feature sets corresponding to the plurality of protein candidate sites; and training a classifier using the plurality of feature sets to classify protein candidate sites for amenability for drug-discovery.

In some embodiments, a fourth non-transitory computer-readable storage medium, for training a classifier for identifying protein candidate sites, is provided, the fourth non-transitory computer-readable storage medium storing instructions configured to be executed by a system comprising one or more processors to cause the system to: receive a corpus of training data comprising data regarding a plurality of protein candidate sites; generate, based on the training data, a plurality of feature sets corresponding to the plurality of protein candidate sites; and train a classifier using the plurality of feature sets to classify protein candidate sites for amenability for drug-discovery.

In some embodiments, a fifth system, for characterizing protein candidate sites, is provided, the fifth system comprising one or more processors configured to cause the fifth system to: receive data comprising a set of protein candidate sites and corresponding metadata regarding experimental data associated with respective sites of the set of candidate sites; for each protein candidate site of the set of protein candidate sites, determine, based on the received data: a number of times that one or more peptides associated with the respective protein candidate site was observed across one or more experimental iterations; and a number of experimental iterations in which one or more peptides associated with the respective protein candidate site was observed; select a subset of the received data, wherein the selection is based on the number of times that the one or more peptides were observed across the one or more experimental iterations and on the number of experimental iterations in which the one or more peptides were observed, wherein the subset of the received data represents a subset of the set of protein candidate sites; and generate and store a characterization of the subset of protein candidate sites, wherein the characterization characterizes amenability of the protein candidate sites for drug-discovery.

In some embodiments of the fifth system, generating the characterization comprises: generating, for each of the respective protein candidate sites in the subset, a respective feature set; and applying a classifier to the respective feature sets in order to generate the characterization.

In some embodiments of the fifth system, the characterization of the amenability for drug-discovery of the protein candidate site comprises a probability of the protein candidate site being reactive.

In some embodiments of the fifth system, the one or more processors are further configured to cause the fifth system to generate and store a ranking of the subset of protein candidate sites, wherein the ranking is based on the characterization of the amenability for drug-discovery for one or more of the protein candidate sites.

In some embodiments of the fifth system, the respective feature set comprises one or more selected from the following: one or more features characterizing frequency of observation of one or more peptides associated with the respective protein candidate site across one or more experimental iterations, as indicated by metadata corresponding to the respective protein candidate site; one or more features characterizing protein abundance for the respective protein candidate site; and one or more features characterizing sequence characteristics associated with the respective protein candidate site.

In some embodiments, a fifth method, for characterizing protein candidate sites, is provided, the fifth method performed at a system comprising one or more processors, the fifth method comprising: receiving data comprising a set of protein candidate sites and corresponding metadata regarding experimental data associated with respective sites of the set of candidate sites; for each protein candidate site of the set of protein candidate sites, determining, based on the received data: a number of times that one or more peptides associated with the respective protein candidate site was observed across one or more experimental iterations; and a number of experimental iterations in which one or more peptides associated with the respective protein candidate site was observed; selecting a subset of the received data, wherein the selection is based on the number of times that the one or more peptides were observed across the one or more experimental iterations and on the number of experimental iterations in which the one or more peptides were observed, wherein the subset of the received data represents a subset of the set of protein candidate sites; and generating and store a characterization of the subset of protein candidate sites, wherein the characterization characterizes amenability of the protein candidate sites for drug-discovery.

In some embodiments, a fifth non-transitory computer-readable storage medium, for characterizing protein candidate sites, is provided, the fifth non-transitory computer-readable storage medium storing instructions configured to be executed by a system comprising one or more processors to cause the system to: receive data comprising a set of protein candidate sites and corresponding metadata regarding experimental data associated with respective sites of the set of candidate sites; for each protein candidate site of the set of protein candidate sites, determine, based on the received data: number of times that one or more peptides associated with the respective protein candidate site was observed across one or more experimental iterations; and a number of experimental iterations in which one or more peptides associated with the respective protein candidate site was observed; select a subset of the received data, wherein the selection is based on the number of times that the one or more peptides were observed across the one or more experimental iterations and on the number of experimental iterations in which the one or more peptides were observed, wherein the subset of the received data represents a subset of the set of protein candidate sites; and generate and store a characterization of the subset of protein candidate sites, wherein the characterization characterizes amenability of the protein candidate sites for drug-discovery.

In some embodiments, a sixth method, of screening potential lead compounds against a protein, is provided, the sixth method comprising: identifying a protein having a protein candidate site characterized as amenable for drug-discovery using any one or more of the first, third, and fifth methods; and testing one or more potential lead compounds for interaction with the protein candidate site of the protein.

In some embodiments, a seventh method, of screening potential lead compounds against a protein, is provided, the seventh method comprising: identifying a protein having a protein candidate site ranked as amenable for drug-discovery by using any one or more of the first, third, and fifth methods; and testing one or more potential lead compounds for interaction with the protein candidate site of the protein.

In some embodiments of the sixth method and/or the seventh method, the interaction of the one or more potential lead compounds with the protein is covalent binding of the one or more potential lead compounds with the protein.

In some embodiments of the sixth method and/or the seventh method, the one or more potential lead compounds covalently bind to the protein candidate site.

In some embodiments of the sixth method and/or the seventh method, the interaction of the one or more potential lead compounds with the protein is non-covalent binding of the one or more potential lead compounds with the protein.

In some embodiments of the sixth method and/or the seventh method, the sixth method and/or the seventh method further comprises selecting a lead compound from the potential lead compounds.

In some embodiments of the sixth method and/or the seventh method, the lead compound is selected based on one or more of binding affinity to the protein candidate site, reaction kinetics with the protein candidate site, extent of covalent modification of the protein candidate site by the lead compound, amount of reaction with off-target sites in the protein, amount of reaction with off-target proteins, agonistic interaction with the protein, antagonist interaction with the protein, or selectivity for the protein candidate site.

In some embodiments of the sixth method and/or the seventh method, the sixth method and/or the seventh method further comprises modifying the lead compound to enhance its binding with the protein.

In some embodiments, a sixth system, for ingesting data from biophysical screening experiments, is provided, the sixth system comprising one or more processors configured to cause the sixth system to: receive experimental metadata comprising spectral data from an experimental data source; in response to receiving the experimental metadata comprising the spectral data, automatically generate, based on the received experimental metadata comprising the spectral data, for each peptide of a plurality of peptides, respective mapping data that maps a respective one of the peptides to one or more proteins of a respective plurality of proteins; automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and store the data set comprising the set of protein candidate sites in a first database.

Any one or more features and/or aspects of the above system, method, and/or non-transitory computer-readable storage medium may be combined in whole or in part with one another and/or with any other feature and/or aspect disclosed herein.

### BRIEF DESCRIPTION OF THE DRAWINGS

FIG. 1 depicts a system for ingesting data from biophysical screening experiments, in accordance with some embodiments.

FIGS. 2A and 2B depict a flow chart describing a method of ingesting data from biophysical screening experiments, in accordance with some embodiments.

FIG. 3 shows MS/MS sampling over the elution of a peptide in an LC/MS/MS experiment, and illustrates acquisition of a spectral count for a peptide.

FIG. 4 depicts a flow chart describing a method of characterizing protein sites, in accordance with some embodiments.

FIG. 5 depicts a flow chart describing a method of training a classifier for identifying protein sites, in accordance with some embodiments.

FIG. 6 depicts a flow chart describing a method of characterizing protein sites, in accordance with some embodiments.

FIG. 7 depicts a computer, in accordance with some embodiments.

### DETAILED DESCRIPTION

Described herein are exemplary embodiments of systems, methods, and techniques for ingesting biophysical screening data and creating a database of protein sites, along with related techniques. The systems, methods, and techniques disclosed herein may address the problems and shortcomings of known systems as described above.

Described herein are exemplary embodiments of systems, methods, and techniques for analyzing data associated with protein sites to assess amenability of the sites for drug discovery, along with related techniques. As used herein, the term “amenability for drug discovery” may refer to the extent to which a protein site is able to adopt a conformation to recognize and bind a small molecule in a covalent or non-covalent mode, thereby enabling small-molecule drug discovery. The systems, methods, and techniques disclosed herein may address the problems and shortcomings of known systems as described above.

### Definitions

The term “peptide” refers to two or more amino acids joined by amide bonds.

The term “polypeptide” refers to a peptide of about 15 or more amino acids in length. “Polypeptide” includes wild-type proteins, protein isoforms, protein mutants, protein aggregates, and proteins that have been modified post-translationally.

The term “protein” refers to polypeptides of about 20 or more amino acids in length. “Proteins” includes wild-type proteins, protein isoforms, protein mutants, protein aggregates, and proteins that have been modified post-translationally.

“Protein isoforms” are proteins derived from a single gene or a single gene family, but which vary in primary sequence due to alternative mRNA splicing.

A “protein mutant” is a protein that differs from its normally occurring sequence by the deletion, insertion, and/or change of one or more amino acids.

A “site” in a polypeptide or a protein refers to a specifically identified amino acid in the polypeptide or protein.

A protein “candidate site” refers to one or more specifically identified amino acids in a protein that are able to recognize and bind a small molecule covalently.

The term “about” used with numerical values in any of the embodiments disclosed herein indicates that both the value specified, as well as values reasonably close to the value specified, such as +/−10%, are included. For example, the phrase “about 20” indicates “20+/−2”, which includes a description of the value 20 itself, as well as values between 18 and 22.

As used herein, the singular forms “a”, “an”, and “the” include plural references unless indicated otherwise or the context clearly dictates otherwise.

This application refers variously to “data stores” as well as to “databases.” It will be understood by a person of skill in the art that any data store referred to herein may be provided as a database and that any database referred to herein may be provided as any other form of computer-readable data store. Various different embodiments of computer-readable storage mediums may be used, without departure from the systems and techniques disclosed herein, for any database, data store, or other computer-readable storage medium referenced herein.

### Exemplary System

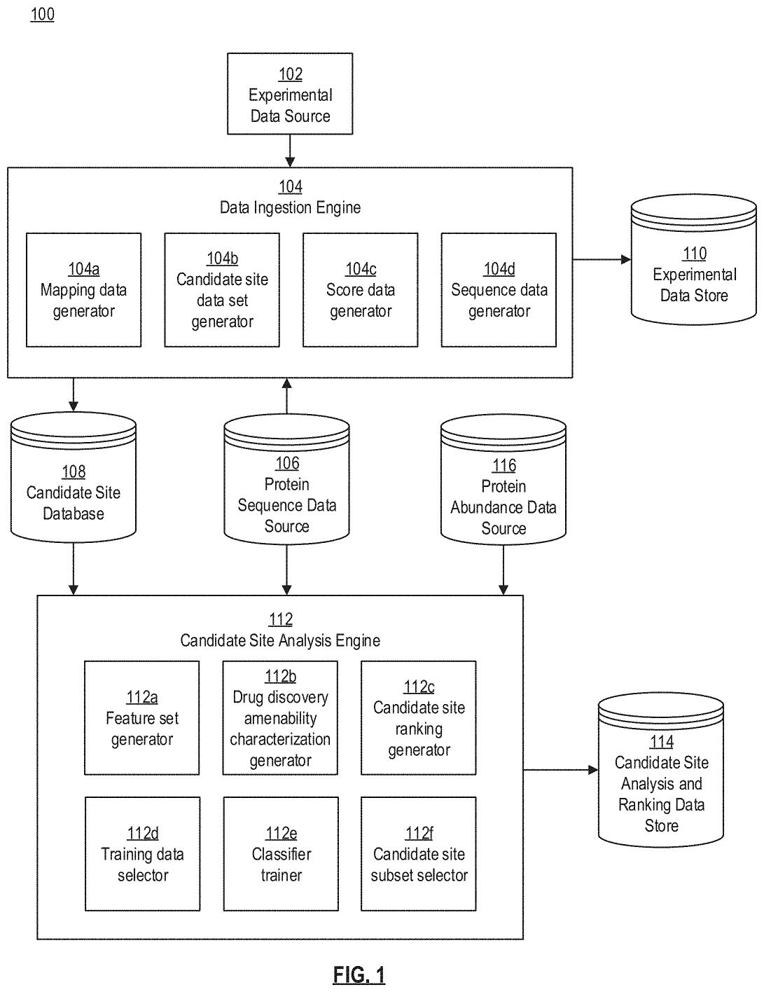

FIG. 1 depicts a system 100 for ingesting data from biophysical screening experiments and for analyzing data associated with protein sites to assess amenability of the sites for drug discovery, in accordance with some embodiments. As described herein, system 100 may perform any one or more of the methods or techniques disclosed herein, and may accordingly address one or more of the needs identified above. As described below in detail, system 100 may provide a computerized system for automatically receiving and processing data from a plurality of biophysical screening experiments, such as spectral data from cell-based biophysical screening experiments. System 100 may be configured to process the received data (and any associated metadata) for ingestion of the data for storage in one or more databases, including by processing the data by comparing it to protein sequence reference data. System 100 may process the received experimental data in order to generate a data set representing one or more protein candidate sites for storage in a database, wherein the protein candidate sites may be sites that are believed, based on the received experimental data, to be reactive and/or amenable for drug discovery. As used herein, the term “amenability for drug discovery” may refer to the extent to which a protein candidate site is able to adopt a conformation to recognize and bind a small molecule in a covalent or non-covalent mode, thereby enabling small-molecule drug discovery. In some embodiments, a small molecule has a molecular weight of 1000 daltons or less. In some embodiments, a small molecule has a molecular weight of about 600 daltons or less. In some embodiments, a small molecule has a molecular weight of about 500 daltons or less. In some embodiments, a small molecule has a molecular weight between about 200 daltons and 1000 daltons, between about 200 daltons and about 600 daltons, or between about 200 daltons and about 500 daltons. System 100 may be configured to update the database of protein candidate sites in accordance with new experimental data received and/or in accordance with detecting an update to protein sequence reference data. In this manner, system 100 may be configured to generate and maintain a database of protein candidate sites.

Biophysical screening experiments include, but are not limited to, cell-based screening experiments, experiments run using one or more purified proteins, experiments run using one or more recombinant proteins, and experiments run in vivo, in vitro, or in situ. Cell-based screening experiments include, but are not limited to, experiments run with individual whole cells, cellular systems such as cell cultures, primary cells, immortalized cells, cell co-culture mixtures, organotypic cell cultures, tissue cultures, tissue samples, cell lysates, and tissue homogenates.

As described herein, system 100 may perform any one or more of the methods or techniques disclosed herein, and may accordingly address one or more of the needs identified above. As described below in detail, system 100 may provide a computerized system for automatically receiving protein candidate site data, analyzing and processing the received data, and generating outputs ranking and/or characterizing the received data to identify protein candidate sites that are believed to be most amenable for drug discovery. System 100 may be configured to process the protein candidate site data (in some embodiments, along with any associated metadata) by applying one or more algorithms to select a subset of the data and/or to rank sites within the selected subset. In some embodiments, system 100 may be configured to train one or more machine-learning algorithms for characterization and/or ranking of the candidate site data in order to identify candidate sites that are suspected to be amenable for drug discovery. In some embodiments, system 100 may be configured to apply one or more machine learning algorithms to the candidate site data in order to identify candidate sites that are determined to be likely to be amenable for drug discovery; application of a machine-learning algorithm by system 100 may, in some embodiments, comprise generation of a feature set representing a candidate site such that the feature set can be used as input for a machine learning classifier.

As shown in FIG. 1, system 100 may comprise experimental data source 102, data ingestion engine 104, protein sequence data source 106, candidate site database 108, experimental data store 110, candidate site analysis engine 112, protein abundance data source 116, and candidate site analysis and ranking data store 114. Each of the components of system 100 is described, by way of reference to some embodiments, in further detail below.

Experimental data source 102 may comprise any one or more computer systems or computer system components configured to store and/or transmit data from one or more biophysical screening experiments. As shown in FIG. 1, experimental data source may be communicatively coupled (e.g., by wired or wireless network communication) with data ingestion engine 104, and may be configured to transmit experimental data from one or more biophysical screening experiments to data ingestion engine 104. In some embodiments, experimental data source may comprise one or more computer processors, servers, personal computers, mobile electronic devices, databases, computer-readable mediums, distributed storage systems, distributed processing systems, and/or network communication devices.

In some embodiments, experimental data source 102 may comprise one or more computer systems registered with system 100 and for which system 100 is configured to accept uploads of experimental data, such as a computer system associated with a data source storing experimental data and/or a laboratory generating experimental data. In some embodiments, system 100 may be configured such that one or more participants may register with the system for uploading experimental data; registering with system 100 may comprise transmitting metadata information regarding experimental configuration (optionally, along with additional metadata) to system 100 such that experimental data uploaded by the registered participant system may thereafter be automatically associated with the participant's metadata.

In some embodiments, system 100 may be configured to provide one or more registration graphical user interfaces for registering system participants, wherein a registration interface may provide a plurality of selectable options and/or fields to be filled out by a registrant to indicate metadata to be associated with the registrant. The metadata indicated via inputs to the registration interface may be transmitted to system 100 and may be stored in one or more storage systems associated with system 100, such as candidate site database 108. In some embodiments, the selectable options and/or fields comprise one or more of: the type of probe used, the presence of compounds, the presence of a test compound, the presence of an inhibitor, solution conditions, digestion strategies, incubation times, digestion times, cell lines, type of experiment performed, type of instrument used for the experiment, protocol used for the experiment, the vendor that provided any reagent, solvent, protein, cell line, or other material used in the experiment, and/or date on which an experiment was performed.

Following registration with system 100, experimental data source 102 may be configured to upload experimental data, associated experimental metadata, and/or updated registrant metadata to system 100, such as by transmitting said information to data ingestion engine 104.

Data ingestion engine 104 may comprise any one or more computer systems or computer system components configured to store and/or receive data (and associated metadata) regarding one or more biophysical screening experiments, to process the received data, and to generate data, based on the received experimental data, a data set representing one or more protein candidate sites for storage in a database.

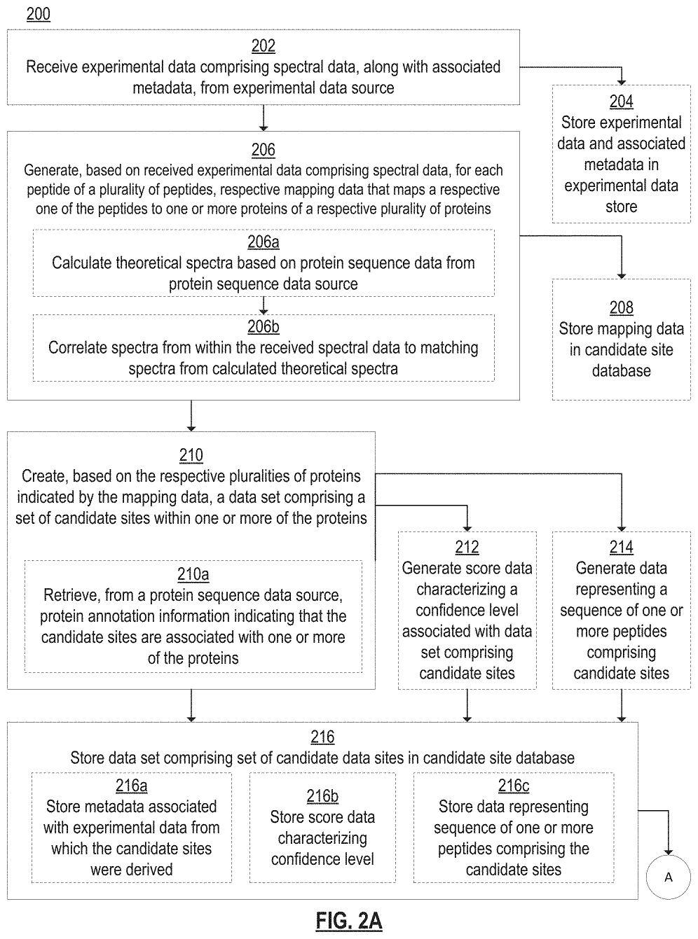

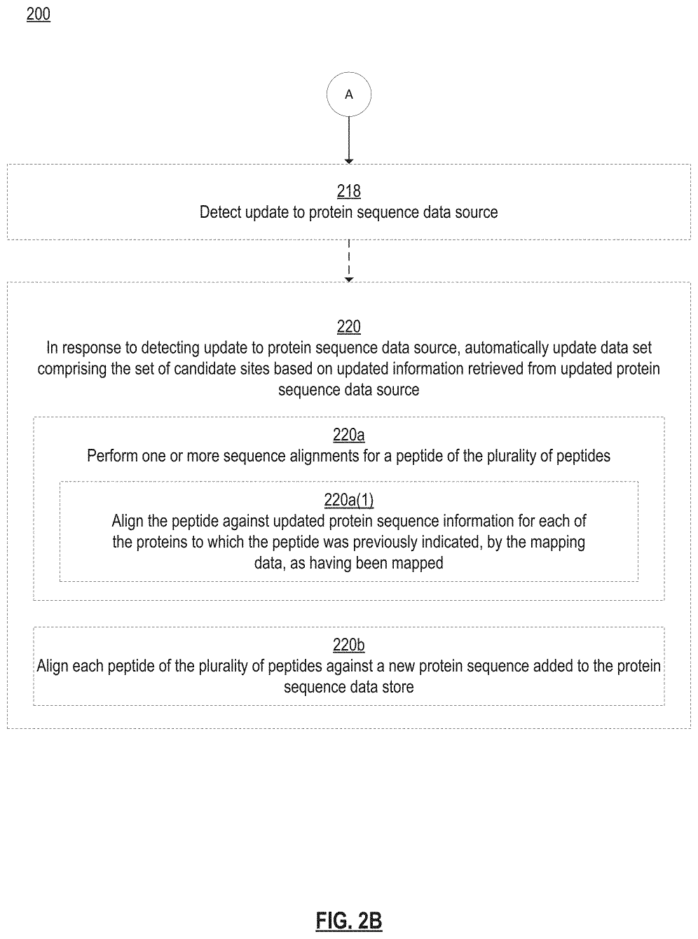

In some embodiments, data ingestion engine 104 may comprise one or more computer processors, servers, personal computers, mobile electronic devices, databases, computer-readable mediums, distributed storage systems, distributed processing systems, and/or network communication devices. Data ingestion engine may be configured to perform one or more methods and/or techniques for data ingestion, data processing, and/or data generation as described herein. As shown, in FIG. 1, data ingestion engine 104 may comprise mapping data generator 104a, candidate site data set generator 10b, score data generator 104c, and sequence data generator 104d, each of which may be configured to perform one or more methods and/or techniques for data ingestion, data processing, and/or data generation as described herein. In some embodiments, one or more different methods/techniques/processes performed by data ingestion engine 104 may be performed by one or more separate processors, separate modules, and/or separate computing systems; in some embodiments, one or more different methods/techniques/processes performed by data ingestion engine 104 may be performed by a same processor or same set of processors. Thus, in some embodiments, one or more of components 104a-104d may represent different processors, different servers, or the like; while, in some embodiments, one or more of components 104a-d may represent different functional capabilities of a single processor or set of processors, single server or set of servers, or the like. Different functionalities that data ingestion 104 (and/or components 104a-104d) may be configured to perform are described in additional detail below with respect to FIGS. 2A-2B and method 200.

Data ingestion engine 104 may be communicatively coupled with experimental data source 102, and may be configured to receive experimental data and/or metadata therefrom, as described above.

Data ingestion engine 104 may be communicatively coupled with protein sequence data source 106, and may be configured to receive protein sequence information therefrom; in some embodiments, protein sequence information received from protein sequence data source 106 may be used by data ingestion engine 104 as protein sequence reference information in processing received experimental data to generate protein candidate site data. In some embodiments, protein sequence data source 106 may comprise one or more computer processors, servers, personal computers, mobile electronic devices, databases, computer-readable mediums, distributed storage systems, distributed processing systems, and/or network communication devices. In some embodiments, protein sequence data source 106 may comprise one or more protein sequence data sets that may be regularly, periodically, and/or intermittently updated; system 100 may be configured such that updates to one or more data sets stored by data source 106 may be detected by system 100 and automatically processed by data ingestion engine 104 to update the protein candidate site data (and/or associated metadata) stored by system 100 as required.

In some embodiments, protein sequence data source 106 may comprise any suitable compendium of protein data. In some embodiments, for each protein in protein sequence data source 106, protein sequence data source 106 may contain information including: one or more protein sequences; one or more protein identifiers (e.g., from protein sequence data source 106 itself and/or from one or more other protein resources); one or more functional annotations, such as catalytic residues, binding sites, post-translationally modified residues, and/or interface regions; cellular localization information; one or more mappings to one or more other protein resources such as Gene Ontology (GO) and/or the Protein Data Bank (PDB); one or more domain annotations; and/or one or more lists of publications supporting evidence for any of the information included in protein sequence data source 106.

In some embodiments, information in protein sequence data source 106 may be accessed directly via a web interface associated with protein sequence data source 106. In some embodiments, information in protein sequence data source 106 may be accessed in bulk, such as by using an ftp associated with protein reference data source 106 to download a large amount of database information, up to and including all information stored in or accessible via the protein sequence data source 106. In some embodiments, information in protein sequence data source 106 may be accessed by an API, such as a REST API, associated with protein sequence data source 106.

In some embodiments, information may be stored on protein sequence data source 106 as a large collection of files describing proteins. Files may be downloaded/retrieved in one or more formats including, for example, tabular (tsv), text, fasta, csv, json, and/or xml.

In some embodiments, protein sequence data source 106 may be curated manually and/or programmatically on an intermittent or periodic basis, such as on a daily basis. As new biological evidence emerges, entries in protein sequence data source 106 may be amended to reflect new insights.

In some embodiments, protein sequence data source 106 may comprise one or more protein sequence data sets that may be regularly, periodically, and/or intermittently updated; system 100 may be configured such that updates to one or more data sets stored by data source 106 may be detected by system 100 and automatically processed by candidate site analysis engine 112 to update analysis and ranking data (and/or associated metadata) stored by system 100 as required.

Data ingestion engine 104 may be communicatively coupled with candidate site database 108, and may be configured to transmit generated protein candidate site data to database 108 for storage thereon. In some embodiments, candidate site database 108 may comprise one or more computer processors, servers, personal computers, mobile electronic devices, databases, computer-readable mediums, distributed storage systems, distributed processing systems, and/or network communication devices. Candidate site database 108 may be configured to receive candidate site data and associated metadata regarding protein candidate sites from data ingestion engine 104 and to store the received data and associated metadata.

In some embodiments, candidate site database 108 may comprise probe information, peptide information, experimental information, and/or protein information. Database 108 may aggregate data from a large body of proteomics experiments performed by any number of parties.

Probe information included in database 108 may comprise information that describes the probe by monoisotopic mass of its adduct. In some embodiments, probe information may also include the probe's name and/or whether the probe is isotopically labeled or not.

Peptide information included in database 108 may comprise a unique numerical identifier for each unique peptide in database 108. For each peptide, database 108 may also include information regarding the sequence of the peptide and whether it has been modified or not. It should be noted that unmodified peptides do not, necessarily, contain a reactive site amenable for drug discovery; however, this data may be used to calculate features and build one or more prioritization scores or other characterizations as described herein. Database 108 may also contain one or more scores that inform the confidence of information such as a spectral match of a peptide. Database 108 may also contain annotation information indicating one or more experiments from which peptide information came, including for example a number of times that particular peptide was observed in the experiment.

Experimental information included in database 108 may comprise information indicating one or more experimental conditions for one or more experiments from which peptide/protein information was derived. Conditions may include the type of probe used, the presence of compounds, the presence of a test compound, the presence of an inhibitor, solution conditions, digestion strategies, incubation times, digestion times, cell lines, type of experiment performed, type of instrument used for the experiment, protocol used for the experiment, the vendor that provided any reagent, solvent, protein, cell line, or other material used in the experiment, and/or date on which an experiment was performed. This experimental metadata may be used to provide insights about the conditions in which certain protein sites were or were not observed.

Data regarding the frequency of observation of a specific peptide can be included in database 108, including spectral count and experiment count. A unique peptide is determined by its amino acid sequence and by the presence or absence of a modification. For example, the peptide ACCCA without any modifying probe is distinct from the peptide ACC*CA, where the star denotes a modification (covalent molecule) at that position. Note that the peptide ACC*CAA is considered distinct from ACC*CA, despite the fact that the sequence of the latter is contained in the former.

The spectral count for a specific, unique peptide may be based on the total number of MS2 spectra identified in liquid chromatography-tandem mass spectrometry (LC/MS/MS) experiments that were matched to that specific, unique peptide. The first mass spectrometer (MS1) is run in data-dependent acquisition mode, and peaks eluting from the chromatograph are fed into MS1. Once at least one ion passes a pre-set intensity threshold, the most intense ion is selected to be fed into the second mass spectrometer (MS2) where it is fragmented for identification. Any given chromatographic peak will feed into MS1 over the period of time required for elution of the peak out of the chromatographic instrument, and MS1 will continue feeding the most intense ion from that chromatographic peak into MS2 over the period of time that at least one ion passes the pre-set intensity threshold. Thus, multiple MS2 spectra may be acquired for a single chromatographic peak. The number of such MS2 spectra acquired is referred to as the “spectral count.”

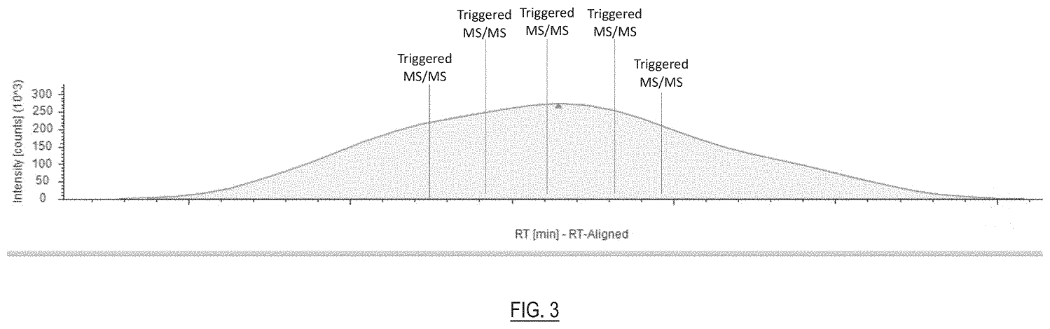

FIG. 3 depicts how the spectral count is acquired for a given peptide. In each precursor scan obtained in data-dependent acquisition (DDA) mode, MS1 ions are triggered for additional fragmentation (MS/MS) based upon signal intensity. Over the course of a single peptide elution (the curve on the graph), the same peptide will be triggered many times for MS/MS data acquisition if it is continually a high-intensity precursor ion, therefore resulting in the acquisition of multiple MS2 spectra for a single unique peptide. The number of spectra acquired is the spectral count. In FIG. 3, five MS/MS acquisitions are triggered, and the spectral count for the peptide illustrated is 5.

It will be appreciated by the skilled artisan that experimental conditions will affect the spectral count; the faster a peak for a given peptide elutes from the chromatograph, the lower the time available for sampling, and the spectral count may be decreased. Conversely, the faster the sampling rate for MS1 during the period when ions above the pre-set intensity threshold are present, the more MS2 spectra will be acquired for any given peak, and the spectral count may be increased. Also, running additional experiments which are analyzed by LC/MS/MS where an MS2 spectrum for a peptide is observed will increase the total spectral count for that peptide. The parameters affecting the spectral count can be stored along with the spectral count itself (for example, chromatograph flow rate, column, and solvents; pre-set ion intensity threshold for triggering MS2 analysis; sampling rate of MS1 while ion intensity is above the pre-set threshold). “Experiment count” refers to the number of experimental iterations in which a specific, unique peptide associated with the respective candidate site were observed. Multiple iterations of an experiment often occur using the exact same conditions (replicates), but for the purpose of calculating the experiment count, each individual experimental iteration is counted.

Spectral count and experiment count can be defined both at the peptide level or the individual site level. At the peptide level, spectral count data is compiled separately for each distinct peptide across alone or more experimental iterations. For spectral counts, the observations at each experimental iteration may be summed together (e.g., in some embodiments, a spectral count may refer to a number of spectra observed in a single experimental iteration, while in some other embodiments a spectral count may refer to a number of spectra observed across multiple (e.g., all) experimental iterations). For experiment counts, each distinct experimental iteration in which that distinct modified peptide was observed is counted. For example, if in experimental iteration 1 the peptide ACC*CA was observed, and 7 spectra were matched to that peptide, and in experimental iteration 2 the peptide ACC*CA was observed, and 5 spectra were matched to that peptide, the spectral count for that peptide is 12, and the experiment count is 2. Note that the experiment count is 2 regardless of whether experimental iteration 1 and experimental iteration 2 were iterations/replicates of the same experiment, or entirely different experiments.

At the site level, the number of observations of distinct peptides where the same residue is modified are combined across all experimental iterations. For example:

Experimental iteration 1: 7 spectra matched to ACC*CA are observed, and 2 spectra matched to ACC*CAA are observed.

Experimental iteration 2: 5 spectra matched to ACC*CA are observed, and 4 spectra matched to ACC*CAA are observed.

Experimental iteration 3: 2 spectra matched to ACC*CAA are observed.

At the site level, for the cysteine residue marked with the asterisk (C*), the spectral count is 20 [i.e., (7+2)+(5+4)+2] (arising from both the peptide ACC*CA and the peptide ACC*CAA), and the experiment count is 3 (the modified site was observed in 3 distinct experimental iterations, regardless of whether the experimental conditions were identical).

At the peptide level, the spectral count is 12 (i.e., 7+5) for the peptide ACC*CA, and the experiment count is 2 (the peptide was seen in Experimental iteration 1 and Experimental iteration 2). The spectral count is 8 (i.e., 2+4+2) for the peptide ACC*CAA, and the experiment count is 3 (the peptide was seen in Experimental iteration 1, Experimental iteration 2, and Experimental iteration 3).

Modification ratios can also be defined for peptides and/or for sites, wherein the modification ratios may be calculated using spectral count and/or experiment count.

A spectral count modification ratio for a given peptide may be defined as the number of spectra matched to the peptide wherein the spectra indicate that the peptide is modified (taken across all experimental iterations), divided by the total number of spectra matched to the peptide whether or not the spectra indicate that the peptide is modified (taken across all experimental iterations). For example, if the spectral count for peptide ACC*CA is 12, and the spectral count for peptide ACCCA (where the lack of an asterisk indicates that no residue was modified) is 3, then the spectral count modification ratio at the peptide level is [12 divided by (12+3)]=0.8.

A spectral count modification ratio for a given site (e.g., at the site level, rather than at the peptide level) may be defined as the number of spectra matched to any peptide containing the given site wherein the spectra indicate that the matched peptide is modified (taken across all experimental iterations), divided by the total number of spectra matched to any peptide containing the residue or site whether or not the spectra indicate that the matched peptide is modified (taken across all experimental iterations). For example, if the spectral count for peptide ACC*CA is 12, the spectral count for peptide ACC*CAA is 8, the spectral count for peptide ACCCA (where the lack of an asterisk indicates that no residue was modified) is 3, and the spectral count for peptide ACCCAA is 17, then the ratio of modification of spectral counts is (12+8) divided by [(12+3)+(8+17)]=0.5.

An experiment count modification ratio for a given peptide may be defined as the number of experimental iterations in which at least one spectra was matched to a distinct modified peptide, divided by the number of experimental iterations in which at least one spectra was matched to a peptide with identical sequence that was either modified or unmodified.

An experiment count modification ratio for a given site (e.g., at the site level rather than at the peptide level) may be defined as the number of experimental iterations in which at least one spectra was matched to any peptide containing a specific modified residue, divided by the number of experimental iterations in which at least one spectra was matched to any peptide that contained that residue, either modified or unmodified.

Data regarding the spectral count modification ratios and/or experiment count modification ratios, for a specific peptide, a specific site, or both, can be included in database 108.

Protein information included in database 108 may comprise information associating proteins with an identifier, which may be an identifier associated with the protein that is used by protein sequence data source 106. Protein information included in database 108 may further comprise a protein's sequence, full description, gene name associated with the protein, and/or species. Protein information included in database 108 may further comprise information regarding a protein's last update date/time from protein sequence data source 106. Protein information included in database 108 may comprise information regarding how each peptide maps to one or more proteins in database 108 or otherwise known to system 100. Protein information included in database 108 may comprise one or more of: one or more protein sequences; one or more protein identifiers (e.g., from protein sequence data source 106 itself and/or from one or more other protein resources); one or more functional annotations, such as catalytic residues, binding sites, post-translationally modified residues, and/or interface regions; cellular localization information; one or more mappings to one or more other protein resources such as Gene Ontology (GO) and/or the Protein Data Bank (PDB); one or more domain annotations; and/or one or more lists of publications supporting evidence for any of the information included in protein sequence data source 106.

Proteins are defined as polypeptides of length about 20 amino acids or longer. In some embodiments, the proteins used in the databases, systems, and methods disclosed herein are of length about 30 amino acids or longer. In some embodiments, the proteins used in the databases, systems, and methods disclosed herein are of length about 40 amino acids or longer. In some embodiments, the proteins used in the databases, systems, and methods disclosed herein are of length about 50 amino acids or longer. In some embodiments, the proteins used in the databases, systems, and methods disclosed herein are of length about 75 amino acids or longer. In some embodiments, the proteins used in the databases, systems, and methods disclosed herein are of length about 100 amino acids or longer. In some embodiments, the proteins used in the databases, systems, and methods disclosed herein are of length about 150, 200, 250, 300, 350, 400, 450, or 500 amino acids or longer.

The largest protein currently known is titin, having a length of about 27,000 to about 35,000 amino acids, depending on the splice isoform. Accordingly, in some embodiments, the proteins used in the databases, systems, and methods disclosed herein are of length about 30, 40, 50, 75, 100, 150, 200, 250, 300, 350, 400, 450, or 500 amino acids to about 35,000 amino acids. In some embodiments, the proteins used in the databases, systems, and methods disclosed herein are of length about 100 amino acids to about 35,000 amino acids. In some embodiments, the proteins used in the databases, systems, and methods disclosed herein are of length about 30, 40, 50, 75, 100, 150, 200, 250, 300, 350, 400, 450, or 500 amino acids to about 20,000 amino acids. In some embodiments, the proteins used in the databases, systems, and methods disclosed herein are of length about 100 amino acids to about 20,000 amino acids. In some embodiments, the proteins used in the databases, systems, and methods disclosed herein are of length about 30, 40, 50, 75, 100, 150, 200, 250, 300, 350, 400, 450, or 500 amino acids to about 10,000 amino acids. In some embodiments, the proteins used in the databases, systems, and methods disclosed herein are of length about 100 amino acids to about 10,000 amino acids. In some embodiments, the proteins used in the databases, systems, and methods disclosed herein are of length about 30, 40, 50, 75, 100, 150, 200, 250, 300, 350, 400, 450, or 500 amino acids to about 5,000 amino acids. In some embodiments, the proteins used in the databases, systems, and methods disclosed herein are of length about 100 amino acids to about 5,000 amino acids. In some embodiments, the proteins used in the databases, systems, and methods disclosed herein are of length about 30, 40, 50, 75, 100, 150, 200, 250, 300, 350, 400, 450, or 500 amino acids to about 1,000 amino acids. In some embodiments, the proteins used in the databases, systems, and methods disclosed herein are of length about 100 amino acids to about 1,000 amino acids.

Data ingestion engage 104 may be communicatively coupled with experimental data store 110, and may be configured to transmit experimental data and/or associated metadata to data store 110 for storage thereon. In some embodiments, data store 110 may comprise one or more computer processors, servers, personal computers, mobile electronic devices, databases, computer-readable mediums, distributed storage systems, distributed processing systems, and/or network communication devices. Data store 110 may be configured to receive experimental data and/or associated metadata from data ingestion engine 104 and to store the received data and associated metadata. In some embodiments, alternatively or additionally, data store 110 may be communicatively coupled with and configured to receive data/metadata directly from experimental data source 102. In some embodiments, data store 110 may be configured to store experimental data and/or metadata the form in which the data is provided before application of one or more data processing techniques by data ingestion engine 104; for example, data store 110 may store “raw data” from experimental data sources while candidate site database 108 may store “processed data” generated by data ingestion engine 104 based on the raw data.

Candidate site database 108 may comprise one or more computer storage mediums configured to store data representing one or more protein candidate sites and/or metadata associated with one or more of the protein candidate sites. In some embodiments, the data and/or metadata may represent an identity of a candidate site, a location of a site within one or more protein sequences, information about one or more known characteristics or aspects of the site (or associated sequence), and/or information about the manner in which the candidate site was ingested into the database. In some embodiments, the data and/or metadata may represent information about a manner in which a candidate site was ingested into and/or selected for inclusion in database 108, such as (a) information about an underlying experiment from which spectral data was derived that was used to select or identify the candidate site and/or (b) information about protein reference data (such as information from protein sequence data source 106 discussed below) used to select or identify the candidate site.

In some embodiments, candidate site database 108 may comprise one or more computer processors, servers, personal computers, mobile electronic devices, databases, computer-readable mediums, distributed storage systems, distributed processing systems, and/or network communication devices. In some embodiments, candidate site database 108 may be communicatively coupled (e.g., by wired or wireless network communication) to candidate site analysis engine 112 (discussed in further detail below) and configured to send and/or receive information thereto and/or therefrom. In some embodiments, candidate site database 108 may be configured to transmit candidate site data and/or associated metadata to candidate site analysis engine 112 for analysis of the data and/or metadata by analysis engine 112 in one or more processes by which analysis engine 112 generates characterizations and/or rankings of candidate sites with respect to the determined/predicted amenability of the candidate sites for drug discovery.

In some embodiments, candidate site database 108 may comprise probe information, peptide information, experimental information, and/or protein information. Database 108 may aggregate data from a large body of proteomics experiments performed by any number of parties.

Probe information included in database 108 may comprise information that describes the probe by monoisotopic mass of its adduct. In some embodiments, probe information may also include the probe's name and/or whether the probe is isotopically labeled or not.

Peptide information included in database 108 may comprise a unique numerical identifier for each unique peptide in database 108. For each peptide, database 108 may also include information regarding the sequence of the peptide and whether it has been modified or not. It should be noted that unmodified peptides do not, necessarily, contain a reactive site amenable for drug discovery; however, this data may be used to calculate features and build one or more prioritization scores or other characterizations as described herein. Database 108 may also contain one or more scores that inform the confidence of information such as a spectral match of a peptide. Database 108 may also contain annotation information indicating one or more experiments from which peptide information came, including for example a number of times that particular peptide was observed in the experiment.

Experimental information included in database 108 may comprise information indicating one or more experimental conditions for one or more experiments from which peptide/protein information was derived. Conditions may include presence of compounds, digestion strategies, incubation and digestion times, cell lines, type of experiment performed, and/or date on which an experiment was performed. This experimental metadata may be used to provide insights about the conditions in which certain sites were or were not observed.

Protein information included in database 108 may comprise information associating proteins with an identifier, which may be an identifier associated with the protein that is used by protein sequence data source 106. Protein information included in database 108 may further comprise a protein's sequence, full description, gene name associated with the protein, and/or species. Protein information included in database 108 may further comprise information regarding a protein's last update date/time from protein sequence data source 106. Protein information included in database 108 may comprise information regarding how each peptide maps to one or more proteins in database 108 or otherwise known to system 100.

Proteins are defined as polypeptides of length about 20 amino acids or longer. In some embodiments, the proteins used in the databases, systems, and methods disclosed herein are of length about 30 amino acids or longer. In some embodiments, the proteins used in the databases, systems, and methods disclosed herein are of length about 40 amino acids or longer. In some embodiments, the proteins used in the databases, systems, and methods disclosed herein are of length about 50 amino acids or longer. In some embodiments, the proteins used in the databases, systems, and methods disclosed herein are of length about 75 amino acids or longer. In some embodiments, the proteins used in the databases, systems, and methods disclosed herein are of length about 100 amino acids or longer. In some embodiments, the proteins used in the databases, systems, and methods disclosed herein are of length about 150, 200, 250, 300, 350, 400, 450, or 500 amino acids or longer.

The largest protein currently known is titin, having a length of about 27,000 to about 35,000 amino acids, depending on the splice isoform. Accordingly, in some embodiments, the proteins used in the databases, systems, and methods disclosed herein are of length about 30, 40, 50, 75, 100, 150, 200, 250, 300, 350, 400, 450, or 500 amino acids to about 35,000 amino acids. In some embodiments, the proteins used in the databases, systems, and methods disclosed herein are of length about 100 amino acids to about 35,000 amino acids. In some embodiments, the proteins used in the databases, systems, and methods disclosed herein are of length about 30, 40, 50, 75, 100, 150, 200, 250, 300, 350, 400, 450, or 500 amino acids to about 20,000 amino acids. In some embodiments, the proteins used in the databases, systems, and methods disclosed herein are of length about 100 amino acids to about 20,000 amino acids. In some embodiments, the proteins used in the databases, systems, and methods disclosed herein are of length about 30, 40, 50, 75, 100, 150, 200, 250, 300, 350, 400, 450, or 500 amino acids to about 10,000 amino acids. In some embodiments, the proteins used in the databases, systems, and methods disclosed herein are of length about 100 amino acids to about 10,000 amino acids. In some embodiments, the proteins used in the databases, systems, and methods disclosed herein are of length about 30, 40, 50, 75, 100, 150, 200, 250, 300, 350, 400, 450, or 500 amino acids to about 5,000 amino acids. In some embodiments, the proteins used in the databases, systems, and methods disclosed herein are of length about 100 amino acids to about 5,000 amino acids. In some embodiments, the proteins used in the databases, systems, and methods disclosed herein are of length about 30, 40, 50, 75, 100, 150, 200, 250, 300, 350, 400, 450, or 500 amino acids to about 1,000 amino acids. In some embodiments, the proteins used in the databases, systems, and methods disclosed herein are of length about 100 amino acids to about 1,000 amino acids.

Protein abundance data source 116 may comprise one or more computer storage mediums configured to store data representing information about protein abundance for one or more proteins. Protein abundance data source 116 may comprise any suitable resource for protein abundances. Protein abundance data source 116 may contain information about protein abundance levels on a per-organism and/or per-cell-line basis. Protein abundance data source 116 may aggregate data from multiple publications into a single, searchable platform. Users may interact with protein abundance data source 116 via a web interface and may be able to download protein abundance data for a particular organism and/or cell line in a tabular format. Updates to protein abundance data source 116 may occur regularly, intermittently, and/or periodically, including for example when new abundance data is published.

In some embodiments, protein abundance data source 116 may comprise one or more computer processors, servers, personal computers, mobile electronic devices, databases, computer-readable mediums, distributed storage systems, distributed processing systems, and/or network communication devices. In some embodiments, protein abundance data source 116 may be communicatively coupled (e.g., by wired or wireless network communication) to candidate site analysis engine 112 (discussed in further detail below) and configured to send and/or receive information thereto and/or therefrom. In some embodiments, protein abundance data source 116 may be configured to transmit protein abundance data and/or associated metadata to candidate site analysis engine 112 for analysis of the data and/or metadata by analysis engine 112 in one or more processes by which analysis engine 112 generates characterizations and/or rankings of candidate sites with respect to the determined/predicted amenability of the candidate sites for drug discovery.

Protein sequence data source 106 may comprise one or more computer storage mediums configured to store data representing information about one or more protein sequences and/or metadata associated therewith. In some embodiments, protein sequence data source 106 may comprise one or more computer processors, servers, personal computers, mobile electronic devices, databases, computer-readable mediums, distributed storage systems, distributed processing systems, and/or network communication devices. In some embodiments, protein sequence data source 106 may be communicatively coupled (e.g., by wired or wireless network communication) to candidate site analysis engine 112 (discussed in further detail below) and configured to send and/or receive information thereto and/or therefrom.

In some embodiments, protein sequence data source 106 may be configured to transmit protein sequence data and/or associated metadata to candidate site analysis engine 112 for analysis of the data and/or metadata by analysis engine 112 in one or more processes by which analysis engine 112 generates characterizations and/or rankings of candidate sites with respect to the determined/predicted amenability of the candidate sites for drug discovery.

In some embodiments, protein sequence data source 106 may comprise any suitable compendium of protein data. In some embodiments, for each protein in protein sequence data source 106, protein sequence data source 106 may contain information including: one or more protein sequences; one or more protein identifiers (e.g., from protein sequence data source 106 itself and/or from one or more other protein resources); one or more functional annotations, such as catalytic residues, binding sites, post-translationally modified residues, and/or interface regions; cellular localization information; one or more mappings to one or more other protein resources such as UniProt, GenBank, Gene Ontology (GO), and/or the Protein Data Bank (PDB); one or more domain annotations; and/or one or more lists of publications supporting evidence for any of the information included in protein sequence data source 106.

In some embodiments, information in protein sequence data source 106 may be accessed directly via a web interface associated with protein sequence data source 106. In some embodiments, information in protein sequence data source 106 may be accessed in bulk, such as by using an ftp associated with protein reference data source 106 to download a large amount of database information, up to and including all information stored in or accessible via the protein sequence data source 106. In some embodiments, information in protein sequence data source 106 may be accessed by an API, such as a REST API, associated with protein sequence data source 106.

In some embodiments, information may be stored on protein sequence data source 106 as a large collection of files describing proteins. Files may be downloaded/retrieved in several format including tabular (tsv), text, and/or xml.

In some embodiments, protein sequence data source 106 may be curated manually and/or programmatically on an intermittent or periodic basis, such as on a daily basis. As new biological evidence emerges, entries in protein sequence data source 106 may be amended to reflect new insights.

In some embodiments, protein sequence data source 106 may comprise one or more protein sequence data sets that may be regularly, periodically, and/or intermittently updated; system 100 may be configured such that updates to one or more data sets stored by data source 106 may be detected by system 100 and automatically processed by candidate site analysis engine 112 to update analysis and ranking data (and/or associated metadata) stored by system 100 as required.

Candidate site analysis engine 112 may comprise any one or more computer systems or computer system components configured to store and/or receive data (and associated metadata) regarding one or more protein candidate sites, to analyze/process the received candidate site data, and/or to generate output data that characterizes and/or ranks the one or more candidate site with respect to a determined/predicted amenability of the candidate site for drug discovery. As described herein, assessment, characterization, and/or ranking of candidate sites may, in some embodiments be based on one or more of (a) candidate site data and/or associated metadata received from candidate site database 108, (b) protein abundance data and/or associated metadata received from protein abundance data source 116, and (c) protein sequence data and/or associated metadata received from protein sequence data source 106.

In some embodiments, candidate site analysis engine 112 may comprise one or more computer processors, servers, personal computers, mobile electronic devices, databases, computer-readable mediums, distributed storage systems, distributed processing systems, and/or network communication devices. Candidate site analysis engine 112 may be configured to perform one or more methods and/or techniques for data analysis, data subset selection, candidate site characterization, feature set generation, machine learning classifier training, and/or candidate site ranking, for example as described herein.

As shown, in FIG. 1, candidate site analysis engine 112 may comprise feature set generator 112a, drug discover amenability characterization generator 112b, candidate site ranking generator 112c, training data selector 112d, classifier trainer 112e, and candidate site subset selector 112f, each of which may be configured to perform one or more methods and/or techniques for data processing/analysis as described herein. In some embodiments, one or more different methods/techniques/processes performed by candidate site analysis engine 112 may be performed by one or more separate processors, separate modules, and/or separate computing systems; in some embodiments, one or more different methods/techniques/processes performed by candidate site analysis engine 112 may be performed by a same processor or same set of processors. Thus, in some embodiments, one or more of components 112a-112f may represent different processors, different servers, or the like; while, in some embodiments, one or more of components 112a-112f may represent different functional capabilities of a single processor or set of processors, single server or set of servers, or the like. Different functionalities that candidate site analysis engine 112 (and/or components 112a-112d) may be configured to perform are described in additional detail below with respect to FIGS. 2-4 and methods 200, 300, and 400.

Candidate site analysis engine 112 may be communicatively coupled with candidate site analysis and ranking data store 114. Data store 114 may comprise any one or more computer-readable storage mediums configured to store candidate site analysis data and/or ranking data characterizing and/or ranking one or more candidate sites. Data stored on data store 114 may be generated by engine 112 and transmitted to data store 114 for storage thereon (in some embodiments along with metadata associated with the analysis data and/or ranking data).

In some embodiments, data store 114 may comprise one or more computer processors, servers, personal computers, mobile electronic devices, databases, computer-readable mediums, distributed storage systems, distributed processing systems, and/or network communication devices.

### Exemplary Methods

FIGS. 2A and 2B depict a flow chart describing a method of ingesting data from biophysical screening experiments, in accordance with some embodiments. Method 200 begins in FIG. 2A and is continued in FIG. 2B. In some embodiments, method 200 may be performed by an electronic system for automatically ingesting data from biophysical screening experiments, such as system 100 described above with reference to FIG. 1.

At block 202, in some embodiments, the system may receive experimental data comprising spectral data, along with associated metadata, from an experimental data source. In some embodiments, the experimental data and associated metadata may be received at data ingestion engine 104. In some embodiments, the experimental data and associated metadata may be received from experimental data source 104. In some embodiments, the experimental data may be received along with associated metadata, for example regarding experimental conditions and parameters; in some embodiments, experimental data and associated metadata may be received separately, such as in separate electronic transmissions.

In some embodiments, the experimental data comprising spectral data may comprise mass spectrometry data. In some embodiments, the experimental data comprising spectral data may comprise tandem mass spectrometry data (MS/MS). In some embodiments, the experimental data comprising spectral data may comprise liquid chromatography/mass spectrometry data (LC/MS). In some embodiments, the experimental data comprising spectral data may comprise liquid chromatography/tandem mass spectrometry data (LC/MS/MS). In some embodiments, the experimental data received by the system may be received before the experimental data is explicitly associated with any peptides and/or proteins. In some embodiments, the experimental data may comprise data from one or more cell-based screening experiments, from one or more in vitro experiments, from one or more in situ experiments, from one or more in vivo experiments, from one or more purified protein experiments, and/or from one or more recombinant protein experiments.

In some embodiments, transmission of experimental data from experimental data sources to an ingestion engine may be enabled by software for communicating with one or more instruments used in the experiment for measuring/creating the data. For example, XCALUBIR software may be used for communicating with instruments and facilitating receipt of experimental data in a raw format. The output of the XCALIBUR software may be raw spectral data as directly measured by an instrument used in the underlying experiment, without any explicit association at this point with any peptides or proteins.

At block 204, in some embodiments, the system may store the received experimental data and associated metadata in an experimental data store. In some embodiments the experimental data store may be data store 110, and the data and/or metadata may be transferred from data ingestion engine 104 to data store 110 for storage thereon. In some embodiments, alternately or additionally, experimental data and/or metadata may be transmitted directly from an experimental data source to an experimental data store, bypassing a data ingestion engine. In some embodiments, user associated with experimental data sources—e.g., users registered to upload experimental data to the system—may save raw experimental data directly to data store 110 themselves (e.g., distinctly from sending the experimental data to ingestion engine 104).

In some embodiments, experimental data and/or metadata may be stored on data store 110 in the same format in which it is received (e.g., it may be stored as “raw data”). In some embodiments, experimental data and/or metadata may be formatted into one or more predefined data formats before being stored on data store 110. In some embodiments, storage of the received experimental data and/or metadata may be performed automatically in response to receiving the data and/or metadata.

Storage of experimental data and/or metadata on a data store such as data store 110 may enable system 100 to retrieve the data as needed at a future time, for example if additional data processing in light of newly received data or in accordance with one or more new or updated data processing algorithms is required.

In some embodiments, data store 110 may be implemented by a third-party file storage service, such as Microsoft One Drive, Amazon Web Services, Google Cloud, or the like.

At block 206, in some embodiments, the system may generate, based on the received experimental data comprising spectral data, for each peptide of a plurality of peptides, respective mapping data that maps a respective one of the peptides to one or more proteins of a respective plurality of proteins. At block 206a, in some embodiments, generating the respective plurality of proteins may comprise calculating theoretical spectra based on protein sequence data from a protein sequence data source. At block 206b, in some embodiments, generating the respective plurality of proteins may comprise correlating spectra from within the received spectral data to matching spectra of the calculated theoretical spectra. In some embodiments, the functionality of block 206, 206a, and/or 206b may be performed by mapping data generator 104a of data ingestion engine 104.

In some embodiments, generating mapping data at block 206 may be performed following receipt of experimental data and/or metadata, including by being performed automatically in response to receiving said experimental data and/or metadata. In some embodiments, generating the mapping data may comprise matching observed spectra as indicated by the experimental data against one or more theoretical spectra, such as theoretical spectra from a collection of calculated theoretical spectra. In some embodiments, system 100 may perform a search of a database containing theoretical spectra data and compare the experimental data to the theoretical spectra data from the search. In some embodiments, system 100 may retrieve protein sequence reference data from a protein sequence data source, such as data source 106, and may calculate one or more theoretical spectra based on the retrieved protein sequence data. Theoretical spectra may be calculated as spectra that may theoretically arise from a protein, or from a fragment of a protein, indicated by the protein reference data. System 100 may then use the calculated theoretical spectra based on the retrieved protein sequence reference information for comparison against the spectral data from the experimental data. System 100 may apply one or more algorithms to assess whether the theoretical spectra are sufficiently similar to the spectra indicated by the experimental data in order for a match to be declared.

In some embodiments, generating the mapping data may be carried out using proteomics software. The proteomics software may be configured to accept input of spectrometry data (e.g., raw mass spectrometry data). The proteomics software may be configured to accept a list of protein sequences; this input may be obtained from protein sequence data source 106.

The proteomics software may be configured to generate output in the form of mapping data, wherein the mapping data represents each peptide represented by the underlying experimental data that was input into the proteomics software, wherein each peptide is mapped to a respective set of proteins, such as protein isoforms (e.g., each peptide mapped to many protein isoforms). The mapping data may represent a “many-to-many” relationship between a plurality of peptides represented by the experimental data and a plurality of proteins to which they are mapped. The mapping data may represent a “many-to-one” relationship between a plurality of peptides mapped to the same protein (e.g., to the same protein isoform). The mapping data may represent a “one-to-many” relationship between a single peptide mapped to a plurality of distinct proteins (e.g., to many distinct protein isoforms).

In some embodiments, the proteomics software applied to generate the mapping data may include one or more of Integrated Proteomics Pipeline (IP2) and Protein Discoverer.

In some embodiments, mapping data generated by system 100 (e.g., generated by mapping data generator 104a) may be automatically associated with experimental data that was uploaded along with or in advance of the experimental data from which the mapping data was derived.

At block 208, in some embodiments, the system may store the generated mapping data in a candidate site database. The candidate site database may, for example, be candidate site database 108. In some embodiments, alternatively or additionally, mapping data may be stored in a database or data store distinct from the candidate site database that stores candidate site information as discussed below.

At block 210, in some embodiments, the system may create, based on the respective one or more proteins of the plurality of proteins indicated by the mapping data, a data set comprising a set of candidate sites within one or more of the proteins. At block 210a, in some embodiments, creating the data set comprising the set of candidate sites within one or more of the proteins may comprise retrieving, from a protein sequence data source, protein annotation information indicating that the candidate sites are associated with one or more of the proteins. In some embodiments, generation of the data representing the sequence of one or more peptides may be performed by candidate site data generator 104b of system 100.

In some embodiments, the system identifies candidate sites by identifying peptides which have been modified by one or more probes. The system can, for example, identify an unmodified peptide mapped to a protein sequence, such as a protein sequence retrieved from a protein sequence data source, and a modified peptide mapped to the same protein sequence based on the increase in mass resulting from the reaction of the probe with the candidate site. In some embodiments, the system identifies the candidate site within the modified peptide based on the probability that a probe will react with a given amino acid. For example, a probe containing an iodoacetamide moiety will preferentially react with cysteine amino acids, and if a peptide modified with such a probe is mapped to a protein which contains a cysteine amino acid, the site of the cysteine amino acid can be identified as the candidate site in the protein.

At block 212, in some embodiments, the system may generate score data characterizing a confidence level associated with the data set comprising the set of candidate sites. In some embodiments, this score data may be generated by a same process or by a related process as the process for generating the mapping data. In some embodiments, this score data may be generated by a same process or by a related process as the process for generating the data set comprising a set of candidate sites. In some embodiments, generation of the score data may be performed by score data generator 104c of system 100.

At block 214, in some embodiments, the system may generate data representing a sequence of one or more peptides comprising one or more of the candidate sites in the set of candidate sites. In some embodiments, generation of the data representing the sequence of one or more peptides may be performed by sequence data generator 104d of system 100.

At block 216, in some embodiments, the system may store the data set comprising the set of candidate data sites in a candidate site database. At block 216a, in some embodiments, storing the data set may comprise storing metadata associated with experimental data from which one or more of the candidate sites in the data set were derived. At block 216b, in some embodiments, storing the data set may comprise storing score data characterizing a confidence level associated with the data set comprising the set of candidate sites. At block 216c, in some embodiments, storing the data set may comprise storing data representing a sequence of one or more peptides comprising one or more of the candidate sites in the set of candidate sites. The candidate site database may, for example, be candidate site database 108.

At block 218 (as shown in FIG. 2B), in some embodiments, the system may detect an update to a protein sequence data source. For example, data ingestion engine 104 may detect that data stored on and/or provided by protein sequence data source 106 has been updated and/or augmented. In some embodiments, data ingestion engine 104 may receive a transmission from protein sequence data source 106 indicating that the reference protein sequence information (and/or associated metadata) stored thereon or provided thereby has been updated. In some embodiments, data ingestion engine 104 may be configured to periodically or intermittently ping protein sequence data source 106 and/or to periodically or intermittently retrieve data from protein sequence data source 106 in order to determine whether data stored thereon or provided thereby has been updated.

In some embodiments, reference protein sequence data and/or protein annotation data may be updated frequently. In some embodiments, updates to protein sequence data and/or protein annotation data may include updates regarding a single-residue change, an insertion of an amino acid, a deletion of an amino acid, a novel polypeptide annotation, a novel protein annotation, merging of two or more protein entries into a single protein entry, and/or a deletion of a protein entry (e.g., due to duplication or lack of biological evidence to support it as a viable protein).

Thus, resources describing mapped proteomics data—such as patents, databases, and/or a publications—may be likely to become increasingly inaccurate over time. Accordingly, the system may be configured to automatically account for updated protein reference information by automatically updating candidate site data accordingly, as described below.

At block 220, in some embodiments, the system may, in response to detecting the update to the protein sequence data source, automatically update the data set comprising the set of candidate sites based on updated information retrieved from the updated protein sequence data source.

At block 220a, in some embodiments, automatically updating the data set may comprise performing one or more sequence alignments for a peptide of the plurality of peptides. At block 220a(1), in some embodiments, performing one or more sequence alignments for a peptide may comprise aligning the peptide against updated protein sequence information for each of the proteins to which the peptide was previously indicated, by the mapping data, as having been mapped.

At block 220b, in some embodiments, automatically updating the data set may comprise aligning each peptide of the plurality of peptides against a new protein sequence added to the protein sequence data store.

In some embodiments, the processes of blocks 220, 220a, 220a(1), and/or 220(b) may be performed by candidate site data set generator 104(b) of system 100.

For example, in some embodiments, after mapping data and/or a data set representing candidate sites derived from the experimental data have already been generated, system 100 may detect an update to the reference data provided by data source 106. In response to detecting said update, system 100 (e.g., data ingestion engine 104) may perform a local sequence alignment for each peptide reflected in the data set representing the candidate sites, wherein the local sequence alignment is performed against all proteins (e.g., for all protein isoforms) that the peptide is mapped against. Alternatively, the local sequence alignment can be performed against only the updated protein sequences.

In some embodiments, in response to detecting an update comprising a new sequence being added to data source 106, one or more peptides reflected in the data set representing the candidate sites (and, in some embodiments, all peptides reflected in the data set representing the candidate sites) may be aligned against the newly added sequence.

The alignment performed by system 100 (e.g., data ingestion engine 104) using new data can result in changes to the candidate site database 108. For example, the alignment against new data can result in additional peptides being associated with a given candidate site. Alternatively, the alignment against new data can result in peptides being removed from association with a given candidate site. Alignment against new data can also result in addition of a candidate site, deletion of a candidate site, or a shift in a candidate site from one amino acid in the protein sequence to a different amino acid in the protein sequence.

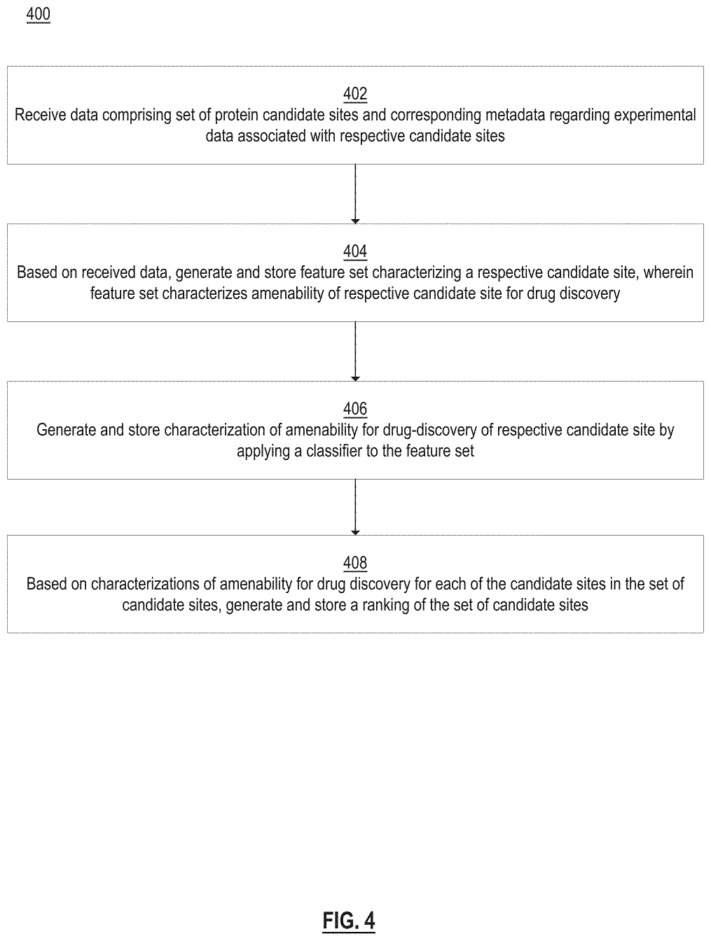

Described below in FIGS. 4-6 are three methods (method 400, method 500, and method 600) that may be performed by a system for protein candidate site analysis and/or ranking, such as system 100.

FIG. 4 depicts a flow chart describing a method 400 of characterizing protein sites, in accordance with some embodiments. In some embodiments, method 400 may be performed by an electronic system for automatically analyzing/ranking protein candidate sites, such as system 100 described above with reference to FIG. 1.

At block 402, in some embodiments, the system may receive data comprising a set of protein candidate sites and corresponding metadata regarding experimental data associated with the respective candidate sites. The data and metadata received by the system may be any data specifying the identity of protein sites, sequences associated with protein sites, known characteristics/properties of protein sites, and/or metadata regarding experimental conditions and/or experimental data from which the protein sites were identified/selected. In some embodiments, the metadata regarding experimental conditions and/or experimental data may include spectral data from one or more biophysical screening experiments that was used to identify the candidate site for further analysis by system 100.

In some embodiments, system 100 may receive said data and/or metadata at analysis engine 112. In some embodiments, analysis engine 112 may receive said data and/or metadata from candidate site database 108; in some embodiments, said data and/or metadata from any other suitable local or remote data source.

At block 404, in some embodiments, based on the received data, the system may generate and store a feature set characterizing a respective candidate site from the set of candidate sites, wherein the feature set characterizes amenability of the respective candidate site for drug discovery. In some embodiments, amenability for drug discovery refers to amenability for discovery of drugs that act by covalent bonding. In some embodiments, amenability for drug discovery refers to amenability for discovery of drugs that act by non-covalent bonding. In some embodiments, generating said feature set may be performed by feature set generator 112a of analysis engine 112.

In some embodiments, system 100 may be configured to use the received candidate site data and/or associated metadata in order to generate one or more feature sets that characterize a respective candidate site with respect to the amenability of the candidate site for drug discovery. In some embodiments, a feature set may include data representing a plurality of features of the candidate site. Each of the plurality of features may represent a different characteristic, a different property, or different information about the candidate site. In some embodiments, the feature set may have a predefined data structure by which certain data (e.g., certain fields, bits, strings, blocks, etc.) represent predefined features in the feature set.

A site residue may be observed with or without a modification by a probe/compound in a given experiment. The same site may be observed multiple times in the same experiment (e.g. same peptide observed multiple times, different peptides containing the site). One or more features may be based on this information and/or based on information from across multiple experiments.

In some embodiments, the feature set may be configured to be human-readable and may itself be used to assess and represent the amenability of the represented candidate site for drug discovery. In some embodiments, the feature set may be configured to be machine-readable and may be configured to be input into one or more analysis algorithms, such as a machine learning classifier, for classification of the candidate site represented by the feature set with regard to determined/predicted amenability for drug discovery. In some embodiments, as described in additional detail below, a machine learning classifier or other algorithm may process a feature set in order to generate output data that characterizes, classifies, and/or ranks a determined/predicted amenability for drug discovery of a candidate site.

In some embodiments, one or more features of the feature set may characterize frequency of experimental observations of one or more peptides associated with the respective protein site (e.g., number of experimental observations of a spectrum matched to one or more peptides). Frequency of experimental observation may be determined, for example, based on metadata corresponding to a respective candidate site and indicating observation of one or more associated proteins across one or more iterations of one or more experiments. This metadata may be part of the metadata received from candidate site database 108.

In some embodiments, one or more features may be based on a number of times that one or more peptides associated with a respective protein site was observed on the basis of “spectral count” as defined herein. In some embodiments, one or more features may be based on a number of times that one or more peptides associated with a respective protein site was observed on the basis of “experiment count” as defined herein. For the purposes of these definitions, a unique peptide is determined by its amino acid sequence and by the presence or absence of a modification. For example, the peptide ACCCA without any modifying probe is distinct from the peptide ACC*CA, where the star denotes a modification (covalent molecule) at that position. Note that the peptide ACC*CAA is considered distinct from ACC*CA, despite the fact that the sequence of the latter is contained in the former.

The spectral count for a specific, unique peptide may be based on the total number of MS2 spectra identified in liquid chromatography-tandem mass spectrometry (LC/MS/MS) experiments that were matched to that specific, unique peptide. The first mass spectrometer (MS1) is run in data-dependent acquisition mode, and peaks eluting from the chromatograph are fed into MS1. Once at least one ion passes a pre-set intensity threshold, the most intense ion is selected to be fed into the second mass spectrometer (MS2) where it is fragmented for identification. Any given chromatographic peak will feed into MS1 over the period of time required for elution of the peak out of the chromatographic instrument, and MS1 will continue feeding the most intense ion from that chromatographic peak into MS2 over the period of time that at least one ion passes the pre-set intensity threshold. Thus, multiple MS2 spectra may be acquired for a single chromatographic peak. The number of such MS2 spectra acquired is referred to as the “spectral count.”

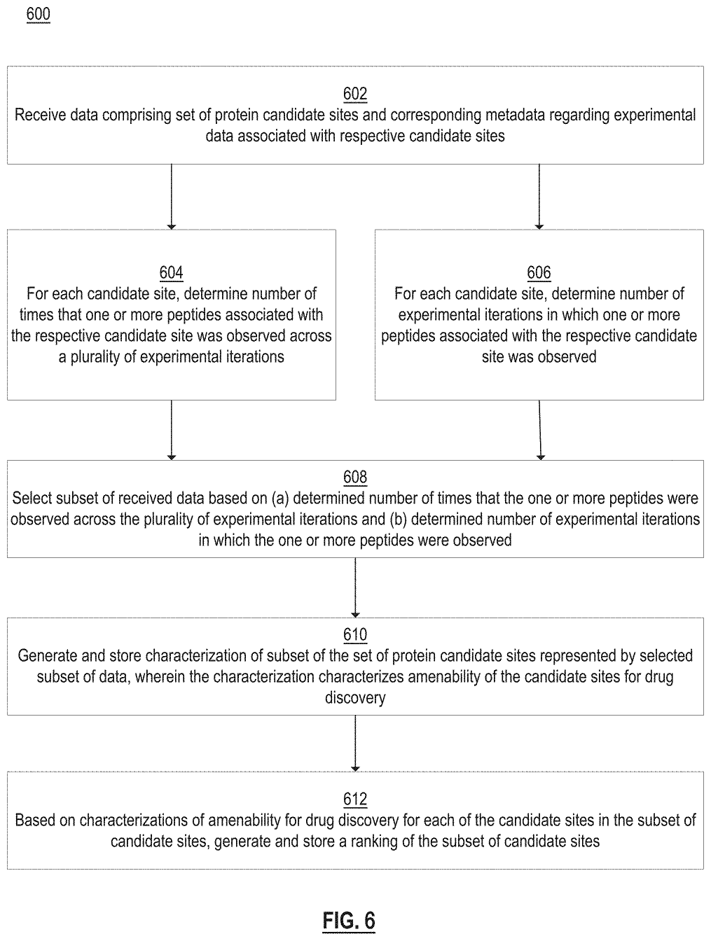

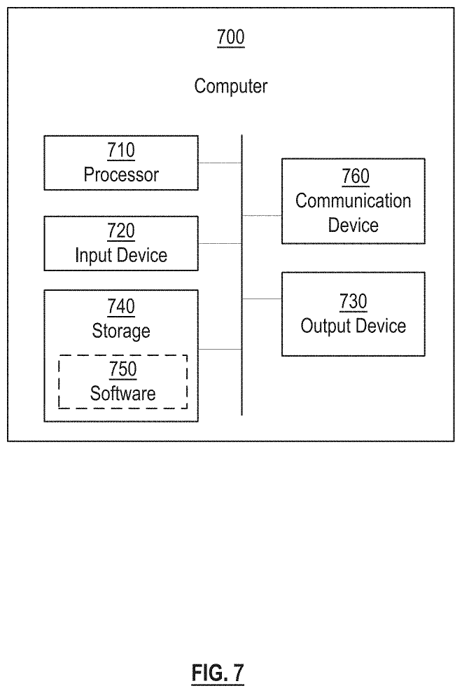

FIG. 6 depicts how the spectral count is acquired for a given peptide. In each precursor scan obtained in data-dependent acquisition (DDA) mode, MS1 ions are triggered for additional fragmentation (MS/MS) based upon signal intensity. Over the course of a single peptide elution (the curve on the graph), the same peptide will be triggered many times for MS/MS data acquisition if it is continually a high-intensity precursor ion, therefore resulting in the acquisition of multiple MS2 spectra for a single unique peptide. The number of spectra acquired is the spectral count. In FIG. 6, five MS/MS acquisitions are triggered, and the spectral count for the peptide illustrated is 5.

It will be appreciated by the skilled artisan that experimental conditions will affect the spectral count; the faster a peak for a given peptide elutes from the chromatograph, the lower the time available for sampling, and the spectral count may be decreased. Conversely, the faster the sampling rate for MS1 during the period when ions above the pre-set intensity threshold are present, the more MS2 spectra will be acquired for any given peak, and the spectral count may be increased. Also, running additional experiments which are analyzed by LC/MS/MS where an MS2 spectrum for a peptide is observed will increase the total spectral count for that peptide. The parameters affecting the spectral count can be stored along with the spectral count itself (for example, chromatograph flow rate, column, and solvents; pre-set ion intensity threshold for triggering MS2 analysis; sampling rate of MS1 while ion intensity is above the pre-set threshold). “Experiment count” refers to the number of experimental iterations in which a specific, unique peptide associated with the respective candidate site were observed. Multiple iterations of an experiment often occur using the exact same conditions (replicates), but for the purpose of calculating the experiment count, each individual experimental iteration is counted.

The features in the feature set can be defined both at the peptide level or the individual site level. At the peptide level, spectral count data is compiled separately for each distinct peptide across one or more experimental iterations. For spectral counts, the observations at each experimental iteration may be summed together (e.g., in some embodiments, a spectral count may refer to a number of spectra observed in a single experimental iteration, while in some other embodiments a spectral count may refer to a number of spectra observed across multiple (e.g., all) experimental iterations). For experiment counts, each distinct experimental iteration in which that distinct modified peptide was observed is counted. For example, if in experimental iteration 1 the peptide ACC*CA was observed, and 7 spectra were matched to that peptide, and in experimental iteration 2 the peptide ACC*CA was observed, and 5 spectra were matched to that peptide, the spectral count for that peptide is 12, and the experiment count is 2. Note that the experiment count is 2 regardless of whether experimental iteration 1 and experimental iteration 2 were iterations/replicates of the same experiment, or entirely different experiments.

At the site level, the number of observations of distinct peptides where the same residue is modified are combined across all experimental iterations. For example:

Experimental iteration 1: 7 spectra matched to ACC*CA are observed, and 2 spectra matched to ACC*CAA are observed.

Experimental iteration 2: 5 spectra matched to ACC*CA are observed, and 4 spectra matched to ACC*CAA are observed.

Experimental iteration 3: 2 spectra matched to ACC*CAA are observed.

At the site level, for the cysteine residue marked with the asterisk (C*), the spectral count is 20 [i.e., (7+2)+(5+4)+2] (arising from both the peptide ACC*CA and the peptide ACC*CAA), and the experiment count is 3 (the modified site was observed in 3 distinct experimental iterations, regardless of whether the experimental conditions were identical).

At the peptide level, the spectral count is 12 (i.e., 7+5) for the peptide ACC*CA, and the experiment count is 2 (the peptide was seen in Experimental iteration 1 and Experimental iteration 2). The spectral count is 8 (i.e., 2+4+2) for the peptide ACC*CAA, and the experiment count is 3 (the peptide was seen in Experimental iteration 1, Experimental iteration 2, and Experimental iteration 3).

Modification ratios can also be defined for peptides and/or for sites, wherein the modification ratios may be calculated using spectral count and/or experiment count.

A spectral count modification ratio for a given peptide may be defined as the number of spectra matched to the peptide wherein the spectra indicate that the peptide is modified (taken across all experimental iterations), divided by the total number of spectra matched to the peptide whether or not the spectra indicate that the peptide is modified (taken across all experimental iterations). For example, if the spectral count for peptide ACC*CA is 12, and the spectral count for peptide ACCCA (where the lack of an asterisk indicates that no residue was modified) is 3, then the spectral count modification ratio at the peptide level is [12 divided by (12+3)]=0.8.

A spectral count modification ratio for a given site (e.g., at the site level, rather than at the peptide level) may be defined as the number of spectra matched to any peptide containing the given site wherein the spectra indicate that the matched peptide is modified (taken across all experimental iterations), divided by the total number of spectra matched to any peptide containing the residue or site whether or not the spectra indicate that the matched peptide is modified (taken across all experimental iterations). For example, if the spectral count for peptide ACC*CA is 12, the spectral count for peptide ACC*CAA is 8, the spectral count for peptide ACCCA (where the lack of an asterisk indicates that no residue was modified) is 3, and the spectral count for peptide ACCCAA is 17, then the ratio of modification of spectral counts is (12+8) divided by [(12+3)+(8+17)]=0.5.

An experiment count modification ratio for a given peptide may be defined as the number of experimental iterations in which at least one spectra was matched to a distinct modified peptide, divided by the number of experimental iterations in which at least one spectra was matched to a peptide with identical sequence that was either modified or unmodified.

An experiment count modification ratio for a given site (e.g., at the site level rather than at the peptide level) may be defined as the number of experimental iterations in which at least one spectra was matched to any peptide containing a specific modified residue, divided by the number of experimental iterations in which at least one spectra was matched to any peptide that contained that residue, either modified or unmodified.

In some embodiments, one or more features may be based on a spectral count at the peptide level. In some embodiments, the spectral count may be defined for modified peptides having a given sequence. In some embodiments, the spectral count may be defined for unmodified peptides having a given sequence. In some embodiments, the spectral count may be defined for the sum of modified and unmodified peptides having a given sequence.

In some embodiments, one or more features may be based on a spectral count at the site level. In some embodiments, the spectral count may be defined for modified peptides having a given sequence. In some embodiments, the spectral count may be defined for unmodified peptides having a given sequence. In some embodiments, the spectral count may be defined for the sum of modified and unmodified peptides having a given sequence.

In some embodiments, one or more features may be based on experiment count at the peptide level. In some embodiments, the experiment count may be defined for modified peptides having a given sequence. In some embodiments, the experiment count may be defined for unmodified peptides having a given sequence. In some embodiments, the experiment count may be defined for the sum of modified and unmodified peptides having a given sequence.

In some embodiments, one or more features may be based on experiment count at the site level. In some embodiments, the experiment count may be defined for modified peptides having a given sequence. In some embodiments, the experiment count may be defined for unmodified peptides having a given sequence. In some embodiments, the experiment count may be defined for the sum of modified and unmodified peptides having a given sequence.

In some embodiments, one or more features may be based on spectral count modification ratio at the peptide level.

In some embodiments, one or more features may be based on spectral count modification ratio at the site level.

In some embodiments, one or more features may be based on the experiment count modification ratio at the peptide level.

In some embodiments, one or more features may be based on experiment count modification ratio at the site level.

In some embodiments, one or more features of the feature set may characterize protein abundance information for the respective protein site. Protein abundance information may be determined, for example, based on data received by candidate site analysis engine 112 from protein abundance data source 116.

In some embodiments, protein abundance information may include whole-organism abundance data for different proteins averaged across different cell lines represented in protein abundance data source 116. In some embodiments, protein abundance information may include cell-line-specific abundance data for different proteins averaged across non-tissue cell lines represented in protein abundance data source 116.

In some embodiments, protein abundance information may describe an absolute value for abundance of different proteins described in a whole-organism data set.

In some embodiments, protein abundance information may describe an absolute value for abundance of different proteins described in a cell-line-specific data set.

In some embodiments, protein abundance information may describe a natural logarithm of an absolute value for abundance of different proteins described in a whole-organism data set.

In some embodiments, protein abundance information may describe a natural logarithm of an absolute value for abundance of different proteins described in a cell-line-specific data set.

In some embodiments, one or more features of the feature set may characterize protein sequence characteristics associated with the respective protein site. For example, a protein sequence characteristic may contain any information regarding a protein sequence associated with the respective protein site, such as any sequence in which the site is located. Protein sequence characteristics may include, for example, one or more numbers quantifying positively and/or negatively charged residues around a site characterized by the feature set. Protein sequence characteristics may be determined, in some embodiments, based on protein sequence data received by candidate site analysis engine 112 from protein sequence data source 106. In some embodiments, protein sequence characteristics may include information regarding a number of charged residues associated with the respective protein site.

In some embodiments, features regarding protein sequence characteristics may be generated with respect to a window of a predetermined number of residues on either side of the target site. In some embodiments, the window may be three residues on either side; in some embodiments, the window may be four residues on either side; in some embodiments, the window may be five residues on either side. In some embodiments, the window may be 6, 7, 8, 9, 10, 11, 12, 13, 14, 15, 16, 17, 18, 19, or 20 residues on either side In some embodiments, features regarding protein sequence characteristics may be generated based on the number of positively charged residues (e.g. Arg, Lys) in the window, the number of negatively charged residues (e.g. Glu, Asp) in the window, and/or and the net charge (number of positives—number of negatives) in the window. In some embodiments, different sequence features may be generated assessing the same information over different window lengths (e.g., three residues versus five residues) and may both be simultaneously used in the same feature set.

In some embodiments, the feature set may include one or more features characterizing additional aspects of experimental observation, distinct from number of observations, of one or more peptides associated with the respective protein. For example, one or more features may characterize a number of experiments, indicated by metadata corresponding to the respective protein site, that include one or more peptides in a modified or unmodified state.

In some embodiments, the feature set may include one or more features characterizing predicted secondary structure of a protein site and/or its nearby residues.

In some embodiments, the feature set may include one or more features characterizing observed secondary structure of a protein site and/or its nearby residues.

In some embodiments, the feature set may include one or more features characterizing sequence-based amino acid propensities of a peptide, polypeptide, or protein.

In some embodiments, the feature set may include one or more features from a position-specific scoring matrix of a protein site and/or its nearby residues.

In some embodiments, the feature set may include one or more features characterizing a length (e.g., an average length) of a peptide that includes the site represented by the feature set.

In some embodiments, the feature set may include one or more features characterizing sequence conservation across families for a protein site and/or its nearby residues. In some embodiments, the families for a protein site and/or its nearby residues are protein families within a single organism, such as humans. In some embodiments, the families for a protein site and/or its nearby residues are protein families within a single taxonomic order, such as primates. In some embodiments, the families for a protein site and/or its nearby residues are protein families within a single taxonomic class, such as mammals. In some embodiments, the families for a protein site and/or its nearby residues are protein families within a single taxonomic phylum, such as chordates. In some embodiments, the families for a protein site and/or its nearby residues are protein families within a single kingdom, such as animalia. In some embodiments, the families for a protein site and/or its nearby residues are protein families within a single domain, such as eukarya. In some embodiments, the families for a protein site and/or its nearby residues are protein families across all domains, encompassing eukarya, archaea, and bacteria.

In some embodiments, the feature set may include one or more features characterizing co-evolutionary information, such as information regarding pairwise co-evolution.

In some embodiments, the feature set may include one or more features characterizing one or more residue counts in a region around a site represented by the feature set. In some embodiments, the feature characterizing one or more residues is a predicted secondary structure. In some embodiments, the feature characterizing one or more residues is an observed secondary structure.

In some embodiments, a residue count may comprise a count of polar residues. In some embodiments, a residue count may comprise a count of apolar residues. In some embodiments, a residue count may comprise both a count of polar residues and a count of apolar residues.

In some embodiments, a residue count may comprise a count of aromatic residues. In some embodiments, a residue count may comprise a count of non-aromatic residues. In some embodiments, a residue count may comprise both a count of aromatic residues and a count of non-aromatic residues.

In some embodiments, the feature set may include one or more features characterizing solvent-accessible surface area associated with a site represented by the feature set. In some embodiments, solvent-accessible surface area information may comprise predicted solvent-accessible surface area information. In some embodiments, solvent-accessible surface area information may comprise observed solvent-accessible surface area information (e.g., as calculated using a spherical probe). At block 406, in some embodiments, the system may generate and store a characterization of amenability for drug discovery of the respective protein site by applying a classifier to the generated feature set. In some embodiments, the classifier may be a machine-learning algorithm configured to classify protein sites based on determined/predicted amenability for drug discovery. In some embodiments, the characterization may comprise a binary characterization, e.g., indicating whether or not a site is determined to be amenable for drug discovery. In some embodiments, the characterization may comprise a score (e.g., a numerical score) quantifying the determined likely amenability for drug discovery of the site. In some embodiments, the characterization may comprise a characterization and/or quantification of a probability (e.g., a number expressed as a percentage) that the site is amenable for drug discovery. In some embodiments, the characterization may comprise a characterization and/or quantification of reactivity. In some embodiments, the characterization may comprise a characterization and/or quantification of reactivity, in which the system is trained to predict experimentally determined reactivity. Reactivity can be determined experimentally using assays such as iMS, NMR, or MS/MS reactivity assessment, for example in conjunction with a nucleophile reference molecule such as iodoacetamide (IA), reduced glutathione (GSH), aniline, or butylamine. In some embodiments, the characterization may comprise a characterization and/or quantification of a probability (e.g., a number expressed as a percentage) that the site is reactive.

In some embodiments, a classifier or other algorithm applied to assess amenability of a site for drug discovery may provide one or more additional functionalities in addition to generating an output comprising an assessment/characterization of protein sites.

In some embodiments, said additional functionalities may include cross-validation functionality that enables partitioning of training sets and training of a classifier into each partition.

In some embodiments, said additional functionalities may include functionality to consolidate results in cross-validation across partitions to obtain a consensus (e.g., best consensus).

In some embodiments, said additional functionalities may include functionality to generate one or more predictions for new data once a classifier has been trained.

In some embodiments, said additional functionalities may include functionality to quantify an importance of one or more individual features in a feature set (e.g., by assigning a score to one or more of the individual features), thus enabling feature selection based on the quantification of individual feature importance.

In some embodiments, said additional functionalities may include functionality to visualize and/or assess performance of the classifier (e.g., AUC calculation, ROC Curve generation).

In some embodiments, said additional functionalities may include functionality to retrieve one or more probabilities calculated by a classifier to assign a label to a novel observation.

In some embodiments, the functionality of block 406 may be performed by drug discovery amenability characterization generator 112b. In some embodiments, output data generated at block 406 (e.g., data representing drug discovery amenability and/or associated metadata) may be transmitted from analysis engine 112 to candidate site analysis and ranking data store 114 for storage thereon.

At block 408, in some embodiments, based on characterizations of amenability for drug discovery for each of the candidate sites in the set of candidate sites, the system may generate and store a ranking of the set of candidate sites, wherein the ranking ranks the candidate sites according to determined/predicted amenability for drug discovery. In some embodiments, the rankings generated by the system may be ranked according to characterization/quantification of amenability for drug discovery, ranked according to quantification of a probability that a site is amenable for drug discovery, and/or ranked according to characterization and/or quantification of a probability (e.g., a number expressed as a percentage) that a site is reactive. In some embodiments, the system may generate one or more ranked lists that may be displayed, stored, and/or transmitted.

In some embodiments, the functionality of block 408 may be performed by candidate site ranking generator 112c. In some embodiments, output data generated at block 408 (e.g., data representing drug discovery rankings, one or more ranked lists, and/or associated metadata) may be transmitted from analysis engine 112 to candidate site analysis and ranking data store 114 for storage thereon.

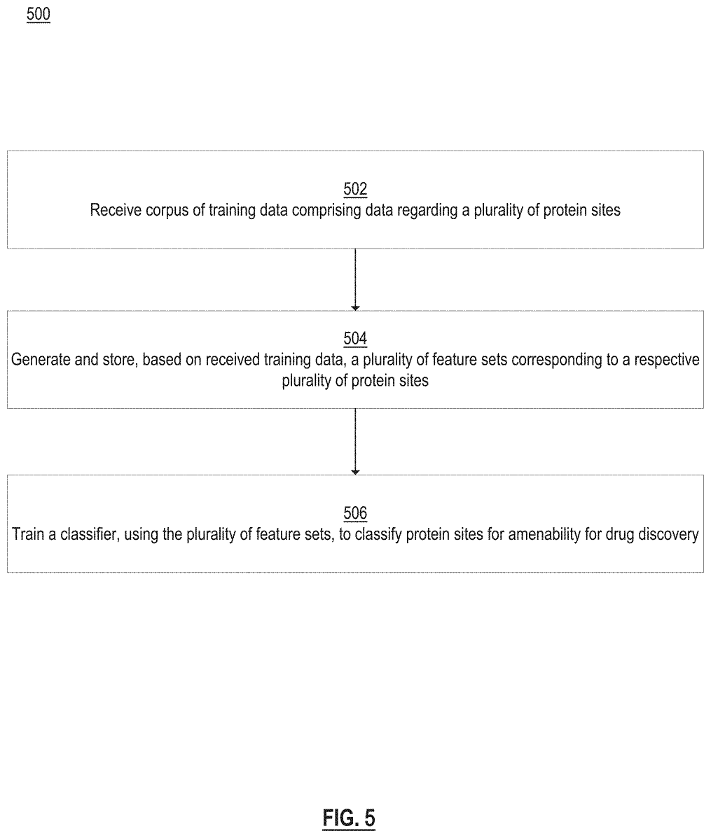

FIG. 5 depicts a flow chart describing a method 500 of training a classifier for identifying protein sites, in accordance with some embodiments. In some embodiments, method 500 may be performed by an electronic system for automatically analyzing/ranking protein candidate sites, such as system 100 described above with reference to FIG. 1.

As described below, method 500 is a method for training a classifier to classify protein sites according to their amenability for drug discovery. In some embodiments, the classifier trained by method 500 may share any one or more characteristics in common with classifiers described above with respect to method 400. In some embodiments, a classifier trained using method 500 may be applied at block 406 of method 400 as described above and/or may be applied at block 610 of method 600 as described below.

At block 502, in some embodiments, the system may receive a corpus of training data regarding a plurality of protein sites. The training data received may be any suitable data set representing protein sites and/or associated sequences, including but not limited to: data specifying the identity of protein sites, sequences associated with protein sites, known characteristics/properties of protein sites, and/or metadata regarding experimental conditions and/or experimental data from which the protein sites were identified/selected. In some embodiments, the training data corpus may share any one or more characteristics in common with the data set (including associated metadata) received above at block 402 of method 400.

In some embodiments, the corpus of training data may comprise labeled data indicating whether one or more portions of the data correspond to a protein site that is known to be amenable for drug discovery or to a protein site that is known to not be amenable for drug discovery. In some embodiments, data labels for the corpus of training data may comprise binary labels (e.g., “amenable” versus “not amenable”) and or quantifications of known amenability (e.g., numerical scores characterizing and quantifying known amenability) for drug discovery.

In some embodiments, a label may indicate (e.g., by a Boolean variable (True/False) whether a site has been annotated as active/catalytic in protein sequence data source 106.

In some embodiments, a label may indicate (e.g., by a Boolean variable (True/False) whether a site has been annotated as a site of post-translational modification in protein sequence data source 106.

In some embodiments, the training data may comprise labels indicating cysteine residue information (in some embodiments, information for cysteine residue labels may be sourced from a protein sequence data source such as protein sequence data source 106). The cysteine residue information may comprise functional and catalytic annotations. In some embodiments, the training data and/or associated labels may comprise any protein sequence data received from a protein sequence data source such as protein sequence data source 106. In some embodiments, the training data may comprise labels indicating non-cysteine residue information (in some embodiments, information for non-cysteine residue labels may be sourced from a protein sequence data source such as protein sequence data source 106), which may be used in a same or similar manner as disclosed herein as cysteine residue labels.

In some embodiments, the training data may comprise labels indicating protein abundance information, such as information retrieved from a protein abundance data source such as protein abundance data source 116.

In some embodiments, the training data may comprise labels indicating information regarding whether a residue is a metal chelation site.

In some embodiments, the training data may comprise labels indicating information regarding experimentally derived isotopic ratios associated with protein sites that may be used to estimate reactivity (e.g., cysteine reactivity).

In some embodiments, the training data may comprise labels indicating information regarding whether a residue is at a termini of alpha-helices.

In some embodiments, the training data may comprise labels indicating information regarding quantified/measured reactivity (e.g., cysteine reactivity) sourced from experimental results (e.g., published results).

In some embodiments, the training data may comprise labels indicating information regarding whether a cysteine is part of a disulfide bridge.

In some embodiments, the training data may comprise labels indicating information regarding quantified reactivity (e.g., cysteine reactivity). In some embodiments, reactivity may be quantified using one or more of: one or more NMR-based reactivity assessment and/or one or more assays (e.g., iMS standardized assays), either of which may be used in conjunction with a nucleophile reference molecule such as GSH, aniline or butylamine.

In some embodiments, the training data may comprise labels indicating information regarding whether a residue constitutes an ubiquitination site.

In some embodiments, selection of training data may be performed on the basis of any one or more labels of the training data. Data sets for training, validation, and/or testing may be selected on the basis of any one or more of the data labels described herein.

In some embodiments, receiving the training data may comprise selecting the plurality of protein sites represented by the training data from a protein sequence data source in accordance with metadata indicating: that the selected protein sites are known to be amenable for drug discovery, that the selected protein sites satisfy one or more isotopic ratio criteria, that the selected protein sites are associated with a numerical score for known drug discovery amenability satisfying one or more predefined threshold criteria, that the selected protein sites are post-translationally modified that the selected protein sites satisfy one or more protein abundance criteria, that the selected protein sites are catalytic, and/or that the associated protein sites satisfy one or more reactivity criteria. Selection of training data may be done on the basis of the labels for the training data. In some embodiments, system 100 may obtain information about the protein sites from a protein sequence data source, and may base the selection of the training data off said information. In some embodiments, the selection of training data may be performed by training data selector 112d of analysis engine 112.

At block 504, in some embodiments, the system may generate and store, based on the received training data, a plurality of feature sets corresponding to a respective plurality of protein sites represented by the training data. In some embodiments, the manner of generating feature sets may share any one or more characteristics in common with the manner of generating feature sets described above with respect to method 400 (e.g., block 404). For example, the feature sets generated at block 504 may represent the same information and may include one or more of the same features as the feature sets generated at block 404 of method 400, with the difference in the operations being that the feature sets generated at block 504 may represent protein sites from the corpus of training data whereas the feature sets generated at block 404 may represent protein candidate sites (e.g., the data received at block 402) for which the system will assess whether (and/or to what extent) the sites are determined/predicted to be amenable for drug discovery. In some embodiments, protein sites represented by the training data (received in method 500) may be known to either be amenable or not amenable for drug discovery, whereas protein sites represented by data received for analysis in method 400 may not be known to be amenable or not amenable for drug discovery before the application of method 400.

In some embodiments, generating said feature sets at block 504 may be performed by feature set generator 112a of analysis engine 112.

In some embodiments, system 100 may be configured to use the received training set protein site data and/or associated metadata in order to generate one or more feature sets that characterize a respective candidate site with respect to the amenability of the candidate site for drug discovery. In some embodiments, a feature set may include data representing a plurality of features of the protein site. Each of the plurality of features may represent a different characteristic, a different property, or different information about the candidate site. In some embodiments, the feature set may have a predefined data structure by which certain data (e.g., certain fields, bits, strings, blocks, etc.) represent predefined features in the feature set.

In some embodiments, the feature set may be configured to be machine-readable and may be configured to be input into one or more analysis algorithms, such as a machine learning classifier, for training of said algorithm for assessing amenability of protein sites for drug discovery.

In some embodiments, one or more features of the feature set may characterize frequency of experimental observation of one or more peptides associated with the respective protein site. Frequency of experimental observation may include any of the spectral counts, experiment counts, spectral count modification ratios, and/or experiment count modification ratios discussed above.

In some embodiments, spectral count and/or experiment count may be determined based on a number of times the one or more peptides were observed with a covalent modification. In some embodiments, spectral count and/or experiment count may be determined based on a number of times the one or more peptides were observed with or without covalent modification.

In some embodiments, one or more features of the feature set may characterize protein abundance information for the respective protein site. Protein abundance information may be determined, for example, based on data received by candidate site analysis engine 112 from protein abundance data source 116, and may include protein abundance information of the type(s) described above with respect to protein abundance data source 116.

In some embodiments, one or more features of the feature set may characterize protein sequence characteristics associated with the respective protein site. For example, a protein sequence characteristic may contain any information regarding a protein sequence associated with the respective protein site, such as any sequence in which the site is located. Protein sequence characteristics may be determined, in some embodiments, based on protein sequence data received by candidate site analysis engine 112 from protein sequence data source 106. In some embodiments, protein sequence characteristics may include information regarding a number of charged residues associated with the respective protein site. In some embodiments, protein sequence characteristics may include any information of the type(s) described above with respect to protein sequence data source 106.

In some embodiments, the feature set may include one or more features characterizing additional aspects of experimental observation, distinct from frequency of observation, of one or more peptides associated with the respective protein. For example, one or more features may characterize a number of experiments, indicated by metadata corresponding to the respective protein site, that include one or more peptides in a modified or unmodified state.

In some embodiments, the feature set may include one or more MS1 (precursor-based) features. For example, the feature set may include one or more features characterizing an area under a curve for an MS1 peak and a height (e.g., maximum intensity) associated with the peak corresponding to a given peptide. In some embodiments, the system may define sum of all areas and/or all intensities at the peptide level and/or at the residue level. In some embodiments, alternatively or additionally, the system may define a ratio of the sum of all areas and/or all intensities of modified peptide (peptide level) or distinct peptides containing modified residue (site level) divided by the sum of all areas and all intensities of peptides modified or unmodified (peptide level) or distinct peptides containing the residue of interest, modified or unmodified (site level).

In some embodiments, the feature set may include one or more features derived from one or more isotopic ratios. For example, the feature set may include one or more features indicating information regarding an average isotopic ratio at the peptide level and/or at the site level either for reactivity-based IsoTOP-ABPP (where heavy and light probes have different concentrations) or competitive IsoTOP-ABPP (where heavy probe and light probe are added to distinct samples, one that is treated with a compound of interest and one that is not). Said ratios may be calculated by taking an area under the curve or the height of an MS1 peak corresponding to peptides modified by either light or heavy probe(s).

At block 506, in some embodiments, the system may train a classifier, using the plurality of feature sets, to classify protein sites for amenability for drug discovery. In some embodiments, a classifier may be trained by applying one or more machine learning models to use the training data to configure the classifier to be able to discriminate between protein site data representing sites that are amenable for drug discovery versus protein site data representing sites that are not amenable for drug discovery. In some embodiments, training the classifier may comprise configuring the classifier to generate any suitable output data regarding classification/analysis/characterization of a protein site, including but not limited to binary classification of the site, characterization of the site regarding amenability for drug discovery, quantification (e.g., assignment of a score) of the amenability for drug discovery of the site, quantification of a probability of the site being amenable for drug discovery, and/or quantification of a probability of the site being reactive. In some embodiments, training the classifier may comprise configuring the classifier to accept feature sets representing one or more candidate protein sites as input and to generate output data in a same or similar manner as discussed above with respect to the output data generated in method 400 (e.g., block 406).

In some embodiments, the system may train the classifier by applying any one or more suitable machine learning models, including but not limited to Support Vector Machines (SVM), Random Forests (RF), and Extreme Gradient Boosting (XGBoost). In some embodiments, the classifier trained may include a CNN, a naïve Bayes system, or a GLM. While the disclosure herein is set forth in terms of using one or more machine-learning-trained classifiers to classify, categorize, characterize, and/or assign one or more quantifications to candidate protein site data, a person of ordinary skill in the art will appreciate, in light of the disclosures made herein, that any suitable algorithm or algorithms (e.g., one or more predictor algorithms or regression algorithms) may be used in a same or similar manner in accordance with the techniques disclosed herein, and that whether or not the algorithm is considered to be a machine-learning-trained classifier may not, in some embodiments, be critical.

In some embodiments, generating said feature sets at block 504 may be performed by feature set generator 112a of analysis engine 112. In some embodiments, a classifier generated or configured at block 504 may be stored on any computer-readable storage media included in or communicatively coupled with analysis engine 112.

FIG. 6 depicts a flow chart describing a method 600 of characterizing protein sites, in accordance with some embodiments. In some embodiments, method 600 may be performed by an electronic system for automatically analyzing/ranking protein candidate sites, such as system 100 described above with reference to FIG. 1.

As described below, method 600 is a method for characterizing protein sites according to their amenability for drug discovery. In some embodiments, method 600 may share any one or more characteristics in common with method 400 as described above; method 600 may differ from method 400 in that method 600 may include one or more steps for selecting a subset of candidate site data before applying one or more characterization, scoring, or ranking algorithms to the subset. That is, rather than applying the overall characterization algorithm to an entire set of candidate site data, method 600 may apply a preliminary “cut-down” step in which the pool of candidate sites is narrowed.

At block 602, in some embodiments, the system may receive data comprising a set of protein candidate sites and corresponding metadata regarding experimental data associated with respective candidate sites. Receipt of data comprising a set of protein candidate sites and corresponding metadata at block 602 may, in some embodiments, share any one or more characteristics in common with receipt of a set of protein candidate sites and corresponding metadata at block 402, as described above with reference to method 400 and FIG. 4. In some embodiments, receipt of data (and associated metadata) at block 602 may comprise receipt of said data (and associated metadata) by analysis engine 112 from one or more of candidate site database 108, protein abundance data source 116, and/or protein sequence data source 106.

As stated above, method 600 may select a subset of candidate site data before applying one or more characterization, scoring, or ranking algorithms to the selected subset. Exemplary steps for subset selection are described below with respect to blocks 604-608.

At block 604, which may follow from block 602, in some embodiments, for each candidate site in the received data, the system may determine a number of times that one or more peptides associated with the respective candidate site was observed across a plurality of experimental iterations (e.g., a “spectral count” as described above).

At block 606, in some embodiments, for each candidate site in the received data, the system may determine number of experimental iterations in which one or more peptides associated with the respective candidate site was observed (e.g., an “experiment count” as described herein).

In some embodiments, one or both of the numbers (e.g., counts) determined at blocks 604 and/or 606 may be determined based on a number of times the one or more peptides were observed with a covalent modification. In some embodiments, one or both of the numbers (e.g., counts) determined at blocks 604 and/or 606 may be determined based on a number of times the one or more peptides were observed with or without covalent modification.

In some embodiments, the determinations made at blocks 604 and/or 606 may be determined based on metadata corresponding to a respective candidate site and indicating observation of one or more associated proteins across one or more iterations of one or more experiments. This metadata may be part of the metadata received by analysis engine 112 from candidate site database 108. In some embodiments, the determinations made at blocks 604 and/or 606 may be performed by analysis engine 112, including by feature set generator 112a and/or candidate site subset selector 112f.

At block 608, in some embodiments, the system may select a subset of the received data based, wherein selection of the subset is based on one or both of (a) the determined number of times that the one or more peptides were observed across the first subset of experimental iterations and (b) the determined number of times that the one or more peptides were observed across the second subset of experimental iterations. The selection of the subset made at 608 may be based on one or both of the determinations made at blocks 604 and 606.

In some embodiments, the system may apply one or more threshold criteria to the numbers determined at blocks 604 and/or 606, and may select only candidate sites who meet one or both threshold criteria. In some embodiments, the system may apply a selection criteria that considers the numbers determined at blocks 604 and 606 in a combined manner, for example by considering a sum of the numbers. In some embodiments, one or both numbers may be weighted before being summed with one another, and the weighted sum may then be considered in making the selection at block 608.

In some embodiments, system 100 may store a record of the selection of the subset of candidate sites, including the selection criteria, one or more selected sites, one or more non-selected sites, and/or associated metadata. In some embodiments, said record of the selection of the subset of candidate sites may be stored in candidate site analysis and ranking data store 114. In some embodiments, the subset selection(s) made at block 608 may be performed by candidate site subset selector 112f.

In some embodiments, system 100 may select the subset of candidate sites based on one or more alternative or additional criteria. For example, in some embodiments, a subset of candidate sites may be selected based on any one or more of the features disclosed herein. In some embodiments, a subset of candidate sites may be selected based on applying a threshold test to the fraction of individual observations of a given site residue in which the site residue was observed to be modified across a plurality of iterations of a single experiment. In some embodiments, a subset of candidate sites may be selected based on applying a threshold test to the fraction of individual observations of a given site residue, across various iterations of a set of multiple experiments, in which the site residue was observed to be modified. In some embodiments, a subset of candidate sites may be selected based on applying a threshold test to the fraction of experiments in which a given site residue was observed as having been modified. Use of one or more of these fractions as a threshold test for selecting a subset of sites may be useful for both cysteine residues and non-cysteine residues.

In some embodiments, only those candidate sites that satisfy one or more selection criteria applied at block 608 may be processed further at blocks 610 and 612, whereas candidate sites not satisfying one or more selection criteria may not be processed further at blocks 610 and 612.

At block 610, in some embodiments, the system may generate and store a characterization of the subset of protein candidate sites represented by the selected subset of data, wherein the characterization characterizes the determined/suspected amenability for drug discovery of the candidate sites in the subset. In some embodiments, generating and storing the characterization of the subset of candidate sites at block 610 may share any one or more characteristics in common with the operations described above with respect to block 406 of method 400. In some embodiments, generating and storing a characterization of the subset of candidate sites may comprise storing data and/or metadata regarding the characterization on candidate site analysis and ranking data store 114. In some embodiments, generating and storing the characterization data (and associated metadata) at block 610 may be performed by drug discovery amenability characterization generator 112b.

In some embodiments, furthermore, because generating and storing characterizations of candidate sites at block 610 may include applying a classifier to feature sets representing the candidate sites, method 600 may also include generating and storing said feature sets based on the subset of candidate sites selected at block 608. In some embodiments, generation of feature sets in method 600 may share any one or more characteristics in common with the operations described above with respect to block 404 of method 400.

In some embodiments, generating and storing a feature set may comprise storing the feature set (and any associated metadata) on any computer-readable storage medium included in or communicatively coupled with analysis engine 112. In some embodiments, generating and storing a feature set as part of method 600 may be performed by feature set generator 112a.

At block 612, in some embodiments, the system may, based on characterizations of the amenability for drug discovery of each of the candidate sites in the subset of candidate sites, generate and store a ranking of the subset of candidate sites. In some embodiments, generating and storing ranking data (along with any associated metadata) based on the characterizations of the subset of selected candidate sites at block 612 may share any one or more characteristics in common with the operations described above with respect to block 408 of method 400.

In some embodiments, generating and storing ranking data comprise storing the ranking data (and any associated metadata) on any computer-readable storage medium included in or communicatively coupled with analysis engine 112, such as candidate site analysis and ranking data store 114. In some embodiments, generating and storing ranking data as part of method 600 may be performed by candidate site ranking generator 112c.

In some embodiments, any one or more of the candidate site analysis methods disclosed herein may be performed in accordance with (e.g., automatically in response to) a determination that a candidate site has a minimum threshold number of data points associated with it. For example, candidate sites in a database with insufficient numbers of data points may not be featurized, classified, and/or ranked; whereas candidate sites in the database with a sufficient numbers of data points may be featurized, classified, and/or ranked as described herein.

In some embodiments, any one or more of the candidate site analysis methods disclosed herein may produce output including a list of cells in which a particular site was observed (which may be useful for assessing a site's presence in a disease context), cellular location, domain and other functional annotations for a particular protein, and/or a PDB structure that contains the site and its corresponding PDB residue index. Output may be provided in table format and/or in an in-depth HTML view that may provide information regarding one or more individual targets represented in the database 108, relating how the sites map to different domain and functional annotations, secondary structure elements, and/or interfacing regions.

### Drug-Discovery Using Systems and Methods

Once candidate sites on proteins have been characterized, classified, or ranked by the systems and methods described herein, the output can then be utilized for lead compound discovery. A lead compound is a compound which is used as a starting point for drug discovery. In view of the high costs of drug discovery and development, identifying a promising lead compound which can be further modified by medicinal chemistry techniques is a valuable step in the process, and excluding compounds which are not suitable leads is also important. In order to identify such lead compounds, many potential lead compounds must be screened against a target of interest, either by wet chemistry methods or by a combination of in silico screening and wet chemistry methods. The systems and methods described herein can guide the skilled artisan to screen against promising targets, and avoid screening against poor targets, thus avoiding the waste of time and resources. The systems and methods described herein can identify avenues for drug discovery that may be completely overlooked by existing methods or not accessible with existing methods, thus opening up new opportunities and new targets for drug discovery. Knowledge concerning proteins with candidate sites that have been characterized, classified, or ranked by the systems and methods described herein is thus of immense value in the drug-discovery process. Accordingly, disclosed herein is a method of screening potential lead compounds against a protein, comprising identifying a protein having a candidate site characterized as amenable for drug-discovery by any system or method disclosed herein, and testing one or more potential lead compounds for interaction with the protein candidate site of the protein. Also disclosed herein is a method of screening potential lead compounds against a protein, comprising identifying a protein having a candidate site ranked as amenable for drug-discovery by any system or method disclosed herein, and testing one or more potential lead compounds for interaction with the protein candidate site of the protein. In some embodiments, the interaction of the one or more potential lead compounds with the protein is covalent binding of the one or more potential lead compounds with the protein. In some embodiments where the interaction of the one or more potential lead compounds with the protein is covalent binding of the one or more potential lead compounds with the protein, the one or more potential lead compounds covalently bind to the protein at the candidate site. In some embodiments, the interaction of the one or more potential lead compounds with the protein is non-covalent binding of the one or more potential lead compounds with the protein.

A lead compound is typically a small molecule. In some embodiments, a small molecule has a molecular weight of 1000 daltons or less. In some embodiments, a small molecule has a molecular weight of about 600 daltons or less. In some embodiments, a small molecule has a molecular weight of about 500 daltons or less. In some embodiments, a small molecule has a molecular weight between about 200 daltons and 1000 daltons, between about 200 daltons and about 600 daltons, or between about 200 daltons and about 500 daltons.

Once a candidate site in a protein of interest is identified, potential lead compounds can be screened for interaction with the protein, and one or more lead compounds can be selected for further refinement. Selection can be based on various criteria, including, but not limited to, the kinetics of reaction of the lead compound with the protein, the extent of covalent modification of the protein by the lead compound, the amount of reaction with off-target sites in the target protein or with off-target proteins, agonistic interaction with the protein, antagonist interaction with the protein, selectivity for the protein, or other criteria.

Once a lead compound for further refinement has been selected, the lead compound can be modified to enhance its binding with the protein target. For lead compounds where structural information is available at the atomic level, functional groups can be introduced which interact with nearby groups on the protein. For example, if a protein has charged groups near the bound lead compound, groups having the opposite charge can be added to the region of the bound lead compound near those charged proteins groups to enhance binding. Other modifications can be made, including, but not limited to, such as an increase or decrease in polar surface area, replacement of groups with near analogs or with isosteric groups, addition of hydrogen-bonding partners to regions of the small molecule close to a counterpart hydrogen-bonding groups on the protein. Such modifications can enhance the binding of the lead compound to the protein and improve its properties, such as increasing its specificity for the protein target.

Refinement of the lead compound can enhance its binding activity to such a degree that covalent binding of the lead compound to the protein target may no longer be required for interaction of the lead compound with the protein target. In such instances, the moiety of the lead compound which forms the covalent bond with the protein target may be removed or modified, such that the lead compound then interacts with the protein target non-covalently, but still binds with sufficient affinity to continue through the drug discovery and development process.

At any point before, during, or after this refinement process for enhancement of binding, or conversion of a covalent lead into a non-covalent lead, other refinements to the lead compound can be made to introduce characteristics desirable for a drug candidate Such other refinements include the “rule of five” characteristics (no more than 5 hydrogen bond donors, no more than 10 hydrogen bond acceptors, molecular mass less than 500, an octanol-water partition coefficient of 5 or less (log P less than 5), number of rotatable bonds, number of aromatic rings, etc.

### Examples

The following procedures are provided as examples of how experimental data can be generated for use in the system and methods disclosed herein. In the examples below, proteins are labeled with test compounds (1 below) or probes (2A and 2B below), fragmented into peptides by enzymatic digestion, then processed by liquid chromatography and tandem mass spectrometry (LC/MS²). The experimental data is then analyzed by comparison to theoretical spectra derived from protein sequences in order to map peptides to proteins.

### Experimental Procedures

1. Labeling Site of Modification on Proteins:

This assay was used to determine amino acid residue(s) in a target protein that is/are covalently modified by a compound of interest. Recombinant protein of interest was diluted to the appropriate concentration in a total volume of 80 μL (average 10 μg or 8 μM) of buffer. The protein was treated with the compound of interest (1-3% DMSO final) for the desired time point. The protein was precipitated by the addition of cold methanol (80/20% v/v methanol/aq.sol.), and centrifuged at 4° C., max speed, for 10 min. The supernatant was aspirated, and the protein pellet was rinsed twice with 100 μL of 0.01M HCl/90% acetone. The protein pellet was then resolubilized with 30 μL of 8M urea/PBS and bath sonicated for 20 min. After the addition of ProteaseMax (0.1% final) and 100 mM ammonium bicarbonate buffer, the samples were reduced with 10 mM TCEP at 60° C. for 30 min. If the protein contained cysteines, they were capped with 12.5 mM iodoacetamide for 30 min. at room temperature in the dark. Samples were then diluted with 120 μL of DPBS and an additional 1.5 μL of 1% ProteaseMax was added to maintain solubility for digestion. The desired digestion enzyme was then added for 18 h digestion at 37° C.

Mass Spectrometry Analysis:

Samples from above were analyzed using a Fusion Lumos (Thermo Fisher Scientific) with the following LC method using water/0.1% formic acid as buffer A, and 80% acetonitrile/20% water/0.1% formic acid for buffer B: 2-5% B over 0.5 minutes, 5-40% B over 22 minutes, then 40-55% B over 1 minute. Data was collected in data-dependent acquisition mode with dynamic exclusion enabled (12 s). One full mass spectrometry (MS¹) scan (300-2000 mass-to-charge ratio (m/z)) was performed at 240 k resolution with an AGC of 4e⁵, followed by MS² scans using HCD 28% collision energy and an AGC target of 2e⁴ detected in the ion trap. Heated capillary temperature was set to 300° C. and the nanospray voltage was set to 2.4 kV.

Data was extracted and analyzed in IP2 v.6.5.5 (Integrated Proteomics Applications, Inc.) as previously described (see Spradlin J. N., et. al. (2019) Harnessing the anti-cancer natural product nimbolide for targeted protein degradation. Nature Chemical Biology 15, 747-755. PMID 31209351) Amino acid residues of interest were searched against compound modification mass as well as methionine oxidation.

isoTOP ABPP Assays

2A. Reactivity Based IsoTOP ABPP Method:

IsoTOP ABPP methodology was used to determine reactivity of cysteine residues in a proteome of interest as previously described in Weerapana, E., et., Quantitative activity profiling predicts functional cysteines in proteomes. Nature 468, 790-5 (2010).) with minor changes.

Briefly, proteome samples can be diluted to either 1 mg/mL or 2 mg/mL for analyses using phosphate buffer saline (PBS). However, 2 mg/mL was utilized for majority of the experiments. Control samples were treated with 100 μM IA alkyne and test samples were treated with 10 μM IA alkyne. IA alkyne concentrations are determined on a per probe basis. Click chemistry was performed by adding 100 μM light-TEV-biotin-azide to each control sample and 100 μM Heavy Tev-biotin tag to each test sample, followed by 1 mM TCEP, 1 mM TBTA and 1 mM CuSO₄. Samples were denatured and resolubilized by heating in 1.2% SDS-PBS to 90° C. for 5 min. The protein solutions were incubated with 170 μL of streptavidin slurry overnight at 4° C. Samples are digested using 0.5 μg/μl sequencing grade trypsin/LysC (Promega). TEV protease cleavage was run for 24 h at 29° C. Peptides were desalted using C18 cartridges from Thermo Fisher and solvent was removed by speed vac. Samples were resolubilized in 20 μL of 0.1% formic acid water to run on the LC/MS as described below.

2B. Competitive isoTOP ABPP Method:

Competitive isoTOP ABPP methodology was used to determine the protein target(s) of a given compound and is based on the method described in literature (see Spradlin J. N., et. al. (2019) Harnessing the anti-cancer natural product nimbolide for targeted protein degradation. Nature Chemical Biology 15, 747-755. PMID 31209351; and references cited therein).

For in situ experiments, treatment concentration and time were determined empirically based on cellular potency and toxicity of the test compound. DMSO concentration was kept at 0.1% final. Both control and test compound treated lysates were treated with 100 μM IA alkyne. Light-TEV-Biotin-tag was clicked onto the control sample, and heavy-TEV-biotin tag was clicked onto the test compound treated samples. The remainder of the protocol is identical to that described in section 2A above.

2C. IsoTOP Mass Spectrometry Analysis:

Samples from Reactivity based IsoTOP ABPP Method and Competitive isoTOP ABPP Method experiments were analyzed using a Fusion Lumos (Thermo Fisher Scientific) with the following LC method using water/0.1% formic acid as buffer A, and 80% acetonitrile/20% water/0.1% formic acid for buffer B: 2% B hold for 1 minute, 2-5% B over 3 minutes, 5-35% B over 74.5 minutes, then 35-45% B over 5 minutes. Data was collected in data-dependent acquisition mode with dynamic exclusion enabled (45 s). One full mass spectrometry (MS¹) scan (375-2000 mass-to-charge ratio (m/z)) was performed at 240 k resolution with an AGC of 1e⁶, followed by MS² scans using HCD 28% collision energy and an AGC target of 1e⁴ detected in the ion trap. Heated capillary temperature was set to 300° C. and the nanospray voltage was set to 2.4 kV.

Data was extracted and analyzed in IP2 v.6.5.5 (Integrated Proteomics Applications, Inc.) as previously described. Cysteine residues were searched with a static modification for carboxyaminomethylation (+57.02146) and up to two differential modifications for methionine oxidation and either the light or heavy TEV tags (+464.28596 or +470.29977, respectively).

### IP2 Data Analysis Additional Details:

Data were extracted in the form of MS¹ and MS² files using Raw Extractor v.1.9.9.2 (Scripps Research Institute) and searched against the Uniprot human database using ProLuCID search methodology in IP2 v.3 (Integrated Proteomics Applications, Inc.). Cysteine residues were searched with a static modification for carboxyaminomethylation (+57.02146) and up to two differential modifications for methionine oxidation and either the light or heavy TEV tags (+464.28596 or +470.29977, respectively). Peptides were required to be fully tryptic peptides and to contain the TEV modification. ProLUCID data were filtered through DTASelect to achieve a peptide false-positive rate below 5%. Only those probe-modified peptides that were evident across two out of three biological replicates were interpreted for their isotopic light to heavy ratios. For those probe-modified peptides that showed ratios greater than two, we only interpreted those targets that were present across all three biological replicates, were statistically significant and showed good quality MS¹ peak shapes across all biological replicates. Light versus heavy isotopic probe-modified peptide ratios are calculated by taking the mean of the ratios of each replicate paired light versus heavy precursor abundance for all peptide-spectral matches associated with a peptide. The paired abundances were also used to calculate a paired sample t-test P value in an effort to estimate constancy in paired abundances and significance in change between treatment and control. P values were corrected using the Benjamini-Hochberg method.

Raw files were uploaded into IP2 and the MS1 and MS2 files were extracted using Raw Converter. MS1 and MS2 files were searched against the Uniprot human proteome database using the ProLuCID algorithm. After completing the ProLuCID search, individual peptides are quantified based on MS1 area under the curve, using the Census algorithm in high resolution (50 ppm) mode.

### Exemplary Embodiments

Below is an enumerated listing of certain embodiments. In some embodiments, any one or more of the features of any one or more of the embodiments below may be combined with any one or more of the other embodiments, even if the dependencies of the embodiments do not explicitly indicate that the embodiments may be combined in such manner. In some embodiments, any one or more of the features of any one or more of the embodiments below may be combined with any one or more features or aspects otherwise disclosed in this application.
1. A system for characterizing protein candidate sites, the system comprising one or more processors configured to cause the system to:

receive experimental data comprising spectral data from an experimental data source;

in response to receiving the experimental data comprising the spectral data, automatically create, based on the experimental data, a data set comprising a set of protein candidate sites within one or more proteins;

for each protein candidate site of the set of protein candidate sites, generate, based on the data set comprising the set of protein candidate sites, a feature set characterizing the respective protein candidate site; and

generate a characterization of the amenability for drug-discovery for one or more of the protein candidate sites by applying a classifier to the respective feature set for the protein candidate site.
2. The system of embodiment 1, wherein:

the one or more processors are further configured to cause the system to automatically generate, based on the received experimental data comprising the spectral data, for each peptide of a plurality of peptides, respective mapping data that maps a respective one of the peptides to a respective plurality of proteins;

automatically creating the data set comprising the set of protein candidate sites is performed based on the respective pluralities of proteins indicated by the generated mapping data; and

the one or more proteins are within in the respective pluralities of proteins.
3. The system of embodiment 2, wherein automatically creating the data set comprising the set of protein candidate sites comprises retrieving, from a protein sequence data source, protein annotation information indicating that the protein candidate sites are associated with one or more of the one or more proteins.
4. The system of embodiment 3, wherein the one or more processors are further configured to cause the system to:

detect an update to the protein sequence data source; and

in response to detecting the update to the protein sequence data source, automatically update the data set comprising the set of protein candidate sites based on updated information retrieved from the updated protein sequence data source.
5. The system of embodiment 4, wherein automatically updating the data set comprising the set of protein candidate sites comprises performing one or more sequence alignments for a peptide of the plurality of peptides.
6. The system of embodiment 5, wherein performing the one or more sequence alignments for the peptide comprises aligning the peptide against updated protein sequence information for each of the proteins to which the peptide was previously indicated, by the mapping data, as having been mapped.
7. The system of any one of embodiments 4-6, wherein automatically updating the data set comprising the set of protein candidate sites comprises aligning each peptide of the plurality of peptides against a new protein sequence added to the protein sequence data store.
8. The system of any one of embodiments 4-7, wherein the updated information retrieved from the updated protein sequence data store comprises one or more of: information indicating a single-residue change, information indicating an insertion of an amino acid, information indicating a deletion of an amino acid, information indicating a novel protein annotation, information indicating merging of two or more protein entries into a single protein entry, and information indicating deletion of a protein entry.
9. The system of any one of embodiments 2-8, wherein the plurality of proteins comprises one or more of a protein isoform and a protein mutant.
10. The system of any one of embodiments 1-9, wherein the one or more processors are further configured to cause the system to store the data set comprising the set of protein candidate sites in a database.
11. The system of any one of embodiments 1-10, wherein the one or more processors are further configured to cause the system to store the generated mapping data in a database.
12. The system of any one of embodiments 1-11, wherein generating the mapping data comprises correlating spectra from within the received spectral data to matching spectra from calculated theoretical spectra.
13. The system of embodiment 12, wherein the one or more processors are further configured to cause the system to calculate the theoretical spectra based on protein sequence data received by the system from a protein sequence data source.
14. The system of any one of embodiments 1-13, wherein the one or more processors are further configured to cause the system to automatically generate and store in a database a sequence of one or more peptides comprising the protein candidate sites.
15. The system of any one of embodiments 1-14, wherein the one or more processors are further configured to cause the system to generate a score characterizing a confidence level associated with at least part of the data set comprising the set of protein candidate sites.
16. The system of any one of embodiments 1-15, wherein the one or more processors are further configured to cause the system to, in response to receiving the experimental data comprising the spectral data, store the experimental data comprising the spectral data in a database.
17. The system of any one of embodiments 1-16, wherein the one or more processors are further configured to cause the system to:

receive metadata specifying experimental conditions for the experimental data source, wherein the data is received via a plurality of predefined fields for experimental condition data types; and

in response to receiving the metadata specifying the experimental conditions, generate and store a record of the experimental conditions in a database.
18. The system of any one of embodiments 1-17, wherein the spectral data comprises mass spectrometry data.
19. The system of any one of embodiments 1-18, wherein the spectral data comprises tandem mass spectrometry data.
20. The system of any one of embodiments 1-19, wherein the spectral data is received by the system before being associated with any peptides or proteins.
21. The system of any one of embodiments 1-20, wherein the data set comprising the set of protein candidate sites comprises indication of a set of candidate residues determined by the system to be potentially modified by promiscuous probes.
22. The system of any one of embodiments 1-21, wherein the experimental data is data generated by one or more of a cell-based screening experiment, an in vitro screening experiment, an in situ screening experiment, an in vivo screening experiment, a purified protein screening experiment, and a recombinant protein screening experiment.
23. The system of any one of embodiments 1-22, wherein the feature set comprises:

one or more features characterizing frequency of observation of one or more peptides associated with the respective protein candidate site across one or more experimental iterations, as indicated by metadata corresponding to the respective protein candidate site;

one or more features characterizing protein abundance for a respective protein comprising the respective protein candidate site; and

one or more features characterizing sequence characteristics associated with the respective protein candidate site.
24. The system of embodiment 23, wherein the one or more features characterizing frequency of observation of the one or more peptides associated with the respective protein candidate site comprise a first feature characterizing a number of times that the one or more peptides associated with the respective protein candidate site were observed across the one or more experimental iterations.
25. The system of embodiment 24, wherein the first feature characterizes a number of times that one peptide associated with the respective protein candidate site was observed across the one or more experimental iterations.
26. The system of any one of embodiments 23-25, wherein the one or more features characterizing frequency of observation of the one or more peptides associated with the respective protein candidate site comprise a second feature characterizing a number of experimental iterations in which the one or more peptides associated with the respective protein candidate site were observed.
27. The system of embodiment 26, wherein the second feature characterizes a number of experimental iterations in which one peptide associated with the respective protein candidate site was observed.
28. The system of any one of embodiments 23-27, wherein the one or more features characterizing frequency of observation of the one or more peptides associated with the respective protein candidate site comprise a third feature characterizing a percentage of experimental observations of the one or more peptides associated with the respective protein candidate site in which the one or more peptides are observed as modified.
29. The system of any one of embodiments 23-28, wherein the third feature characterizes a percentage of experimental observations of one peptide associated with the respective protein candidate site in which the one peptide is observed as modified.
30. The system of any one of embodiments 23-29, wherein the one or more features characterizing frequency of observation of the one or more peptides associated with the respective protein candidate site comprise a fourth feature characterizing a percentage of experiments in which the one or more peptides associated with the respective protein candidate site are observed in which the one or more peptides are observed as modified.
31. The system of embodiment 30, wherein the fourth feature characterizes a percentage of experiments in which one peptide associated with the respective protein candidate site is observed in which the one peptide is observed as modified.
32. The system of any one of embodiments 23-31, wherein the one or more features characterizing protein abundance comprise a fifth feature characterizing protein abundance data retrieved from a protein abundance data source.
33. The system of any one of embodiments 23-32, wherein the one or more features characterizing sequence characteristics comprise a sixth feature characterizing a number of charged residues associated with the respective protein candidate site.
34. The system of any one of embodiments 23-33, wherein the feature set comprises one or more features characterizing additional aspects of experimental observation, distinct from the one or more features characterizing frequency of observation, of one or more peptides associated with the respective protein candidate site.
35. The system of embodiment 34, wherein the one or more features characterizing additional aspects comprise a seventh feature characterizing a number of experimental iterations, indicated by the metadata corresponding to the respective protein candidate site, that include one or more peptides in a modified or unmodified state.
36. The system of any one of embodiments 23-35, wherein the characterization of the amenability for drug-discovery of the protein candidate site comprises a probability of the protein candidate site being reactive.
37. The system of any one of embodiments 23-36, wherein the one or more processors are further configured to cause the system to generate and store a ranking of the set of protein candidate sites, wherein the ranking is based on the characterization of the amenability for drug-discovery for one or more of the protein candidate sites generated by the classifier.
38. A method for characterizing protein candidate sites, the method performed at a system comprising one or more processors, the method comprising:

receiving experimental data comprising spectral data from an experimental data source;

in response to receiving the experimental data comprising the spectral data, automatically creating, based on the experimental data, a data set comprising a set of protein candidate sites within one or more proteins;

for each protein candidate site of the set of protein candidate sites, generating, based on the data set comprising the set of protein candidate sites, a feature set characterizing the respective protein candidate site; and

generating a characterization of the amenability for drug-discovery for one or more of the protein candidate sites by applying a classifier to the respective feature set for the protein candidate site.
39. A non-transitory computer-readable storage medium for characterizing protein candidate sites, the non-transitory computer-readable storage medium storing instructions configured to be executed by a system comprising one or more processors to cause the system to:
   receive experimental data comprising spectral data from an experimental data source;

in response to receiving the experimental data comprising the spectral data, automatically create, based on the experimental data, a data set comprising a set of protein candidate sites within one or more proteins;

for each protein candidate site of the set of protein candidate sites, generate, based on the data set comprising the set of protein candidate sites, a feature set characterizing the respective protein candidate site; and

generate a characterization of the amenability for drug-discovery for one or more of the protein candidate sites by applying a classifier to the respective feature set for the protein candidate site.
40. A system for ingesting data from biophysical screening experiments, the system comprising one or more processors configured to cause the system to:

receive experimental data comprising spectral data from an experimental data source;

in response to receiving the experimental data comprising the spectral data, automatically generate, based on the received experimental data comprising the spectral data, for each peptide of a plurality of peptides, respective mapping data that maps a respective one of the peptides to one or more proteins of a respective plurality of proteins;

automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and

store the data set comprising the set of protein candidate sites in a first database.
41. The system of embodiment 40, wherein the one or more processors are further configured to cause the system to store the generated mapping data in the first database.
42. The system of any one of embodiments 40-41, wherein generating the mapping data comprises correlating spectra from within the received spectral data to matching spectra from calculated theoretical spectra.
43. The system of embodiment 42, wherein the one or more processors are further configured to cause the system to calculate the theoretical spectra based on protein sequence data received by the system from a protein sequence data source.
44. The system of any one of embodiments 40-43, wherein the one or more processors are further configured to cause the system to automatically generate and store in the first database a sequence of one or more peptides comprising the protein candidate sites.
45. The system of any one of embodiments 40-44, wherein the one or more processors are further configured to cause the system to generate a score characterizing a confidence level associated with at least part of the data set comprising the set of protein candidate sites.
46. The system of any one of embodiments 40-45, wherein the one or more processors are further configured to cause the system to:

detect an update to the protein sequence data source; and

in response to detecting the update to the protein sequence data source, automatically update the data set comprising the set of protein candidate sites based on updated information retrieved from the updated protein sequence data source.
47. The system of embodiment 46, wherein automatically updating the data set comprising the set of protein candidate sites comprises performing one or more sequence alignments for a peptide of the plurality of peptides.
48. The system of embodiment 47, wherein performing the one or more sequence alignments for the peptide comprises aligning the peptide against updated protein sequence information for each of the proteins to which the peptide was previously indicated, by the mapping data, as having been mapped.
49. The system of any one of embodiments 46-48, wherein automatically updating the data set comprising the set of protein candidate sites comprises aligning each peptide of the plurality of peptides against a new protein sequence added to the protein sequence data store.
50. The system of any one of embodiments 46-49, wherein the updated information retrieved from the updated protein sequence data store comprises one or more of: information indicating a single-residue change, information indicating an insertion of an amino acid, information indicating a deletion of an amino acid, information indicating a novel protein annotation, information indicating merging of two or more protein entries into a single protein entry, and information indicating deletion of a protein entry.
51. The system of any one of embodiments 40-50 wherein the one or more processors are further configured to cause the system to, in response to receiving the experimental data comprising the spectral data, store the experimental data comprising the spectral data in a second database.
52. The system of any one of embodiments 40-51, wherein the one or more processors are further configured to cause the system to:

receive metadata specifying experimental conditions for the experimental data source, wherein the metadata is received via a plurality of predefined fields for experimental condition data types; and

in response to receiving the metadata specifying the experimental conditions, generate and store a record of the experimental conditions in the first database.
53. The system of any one of embodiments 40-52, wherein the spectral data comprises mass spectrometry data.
54. The system of any one of embodiments 40-53, wherein the spectral data comprises tandem mass spectrometry data.
55. The system of any one of embodiments 40-54, wherein the spectral data is received by the system before being associated with any peptides or proteins.
56. The system of any one of embodiments 40-55, wherein the data set comprising the set of protein candidate sites comprises indication of a set of candidate residues determined by the system to be potentially modified by promiscuous probes.
57. The system of any one of embodiments 40-56, wherein the experimental data is data generated by one or more of a cell-based screening experiment, an in vitro screening experiment, an in situ screening experiment, an in vivo screening experiment, a purified protein screening experiment, and a recombinant protein screening experiment.
58. The system of any one of embodiments 40-57, wherein the plurality of proteins comprises one or more of a protein isoform and a protein mutant.
59. A method for ingesting data from biophysical screening experiments, the method performed at a system comprising one or more processors, the method comprising:

receiving experimental data comprising spectral data from an experimental data source;

in response to receiving the experimental data comprising the spectral data, automatically generating, based on the received experimental data comprising the spectral data, for each peptide of a plurality of peptides, respective mapping data that maps a respective one of the peptides to one or more proteins of a respective plurality of proteins;

automatically creating, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and

storing the data set comprising the set of protein candidate sites in a first database.
60. A non-transitory computer-readable storage medium for ingesting data from biophysical screening experiments, the non-transitory computer-readable storage medium storing instructions configured to be executed by a system comprising one or more processors to cause the system to:

receive experimental data comprising spectral data from an experimental data source;

in response to receiving the experimental data comprising the spectral data, automatically generate, based on the received experimental data comprising the spectral data, for each peptide of a plurality of peptides, respective mapping data that maps a respective one of the peptides to one or more proteins of a respective plurality of proteins;

automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and

store the data set comprising the set of protein candidate sites in a first database.
61. A system for ingesting data from biophysical screening experiments, the system comprising one or more processors configured to cause the system to:

receive experimental metadata comprising spectral data from an experimental data source;

in response to receiving the experimental metadata comprising the spectral data, automatically generate, based on the received experimental metadata comprising the spectral data, for each peptide of a plurality of peptides, respective mapping data that maps a respective one of the peptides to one or more proteins of a respective plurality of proteins;

automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and

store the data set comprising the set of protein candidate sites in a first database.
62. A system for characterizing protein candidate sites, the system comprising one or more processors configured to cause the system to:

receive data comprising a set of protein candidate sites and corresponding metadata regarding experimental data associated with respective protein candidate sites of the set of protein candidate sites;

for each protein candidate site of the set of protein candidate sites, generate, based on the received data, a feature set characterizing the respective protein candidate site, wherein the feature set comprises:
one or more features characterizing frequency of observation of one or more peptides associated with the respective protein candidate site across one or more experimental iterations, as indicated by metadata corresponding to the respective protein candidate site;
one or more features characterizing protein abundance for a respective protein comprising the respective protein candidate site; and
one or more features characterizing sequence characteristics associated with the respective protein candidate site; and

generate a characterization of the amenability for drug-discovery for one or more of the protein candidate sites by applying a classifier to the respective feature set for the protein candidate site.
63. The system of embodiment 62, wherein the characterization of the amenability for drug-discovery of the protein candidate site comprises a probability of the protein candidate site being reactive.
64. The system of any one of embodiments 62-63, wherein the one or more processors are further configured to cause the system to generate and store a ranking of the set of protein candidate sites, wherein the ranking is based on the characterization of the amenability for drug-discovery for one or more of the protein candidate sites generated by the classifier.
65. The system of any one of embodiments 62-64, wherein the one or more features characterizing frequency of observation of the one or more peptides associated with the respective protein candidate site comprise a first feature characterizing a number of times that the one or more peptides associated with the respective protein candidate site were observed across the one or more experimental iterations.
66. The system of embodiment 65, wherein the first feature characterizes a number of times that one peptide associated with the respective protein candidate site was observed across the one or more experimental iterations.
67. The system of any one of embodiments 62-66, wherein the one or more features characterizing frequency of observation of the one or more peptides associated with the respective protein candidate site comprise a second feature characterizing a number of experimental iterations in which the one or more peptides associated with the respective protein candidate site were observed.
68. The system of embodiment 67, wherein the second feature characterizes a number of experimental iterations in which one peptide associated with the respective protein candidate site was observed.
69. The system of any one of embodiments 62-68, wherein the one or more features characterizing frequency of observation of the one or more peptides associated with the respective protein candidate site comprise a third feature characterizing a percentage of experimental observations of the one or more peptides associated with the respective protein candidate site in which the one or more peptides are observed as modified.
70. The system of any one of embodiments 62-69, wherein the third feature characterizes a percentage of experimental observations of one peptide associated with the respective protein candidate site in which the one peptide is observed as modified.
71. The system of any one of embodiments 62-70, wherein the one or more features characterizing frequency of observation of the one or more peptides associated with the respective protein candidate site comprise a fourth feature characterizing a percentage of experiments in which the one or more peptides associated with the respective protein candidate site are observed in which the one or more peptides are observed as modified.
72. The system of embodiment 71, wherein the fourth feature characterizes a percentage of experiments in which one peptide associated with the respective protein candidate site is observed in which the one peptide is observed as modified.
73. The system of any one of embodiments 62-72, wherein the one or more features characterizing protein abundance comprise a fifth feature characterizing protein abundance data retrieved from a protein abundance data source.
74. The system of any one of embodiments 62-73, wherein the one or more features characterizing sequence characteristics comprise a sixth feature characterizing a number of charged residues associated with the respective protein candidate site.
75. The system of any one of embodiments 62-74, wherein the feature set comprises one or more features characterizing additional aspects of experimental observation, distinct from the one or more features characterizing frequency of observation, of one or more peptides associated with the respective protein candidate site.
76. The system of embodiment 75, wherein the one or more features characterizing additional aspects comprise a seventh feature characterizing a number of experimental iterations, indicated by the metadata corresponding to the respective protein candidate site, that include one or more peptides in a modified or unmodified state.
77. A method for characterizing protein candidate sites, the method performed at a system comprising one or more processors, the method comprising:

receiving data comprising a set of protein candidate sites and corresponding metadata regarding experimental data associated with respective protein candidate sites of the set of protein candidate sites;

for each protein candidate site of the set of protein candidate sites, generating, based on the received data, a feature set characterizing the respective protein candidate site, wherein the feature set comprises:
one or more features characterizing frequency of observation of one or more peptides associated with the respective protein candidate site across one or more experimental iterations, as indicated by metadata corresponding to the respective protein candidate site;
one or more features characterizing protein abundance for a respective protein comprising the respective protein candidate site; and
one or more features characterizing sequence characteristics associated with the respective protein candidate site; and

generating a characterization of the amenability for drug-discovery for one or more of the protein candidate sites by applying a classifier to the respective feature set for the protein candidate site.

78. A non-transitory computer-readable storage medium for characterizing protein candidate sites, the non-transitory computer-readable storage medium storing instructions configured to be executed by a system comprising one or more processors to cause the system to:

receive data comprising a set of protein candidate sites and corresponding metadata regarding experimental data associated with respective protein candidate sites of the set of protein candidate sites;

for each protein candidate site of the set of protein candidate sites, generate, based on the received data, a feature set characterizing the respective protein candidate site, wherein the feature set comprises:
one or more features characterizing frequency of observation of one or more peptides associated with the respective protein candidate site across one or more experimental iterations, as indicated by metadata corresponding to the respective protein candidate site;
one or more features characterizing protein abundance for a respective protein comprising the respective protein candidate site; and
one or more features characterizing sequence characteristics associated with the respective protein candidate site; and

generate a characterization of the amenability for drug-discovery for one or more of the protein candidate sites by applying a classifier to the respective feature set for the protein candidate site.
79. A system for training a classifier for identifying protein candidate sites, the system comprising one or more processors configured to cause the system to:

receive a corpus of training data comprising data regarding a plurality of protein candidate sites;

generate, based on the training data, a plurality of feature sets corresponding to the plurality of protein candidate sites; and

train a classifier using the plurality of feature sets to classify protein candidate sites for amenability for drug-discovery.
80. The system of embodiment 79, wherein the training data set comprises cysteine residue information from a protein sequence data source.
81. The system of any one of embodiments 79-80, wherein receiving the training data set comprises selecting the plurality of protein candidate sites from a protein sequence data source in accordance with metadata indicating one of (a) that the protein candidate sites are catalytic and (b) that the protein sites are not catalytic.
82. The system of any one of embodiments 79-81, wherein receiving the training data set comprises selecting the plurality of protein candidate sites from a protein sequence data source in accordance with metadata indicating that the protein candidate sites are post-translationally modified.
83. The system of any one of embodiments 79-82, wherein receiving the training data set comprises selecting the plurality of protein candidate sites from a protein sequence data source in accordance with metadata indicating that the protein candidate sites are amenable for drug discovery.
84. The system of any one of embodiments 79-83, wherein receiving the training data set comprises selecting the plurality of protein candidate sites from a protein sequence data source in accordance with metadata indicating that the protein candidate sites are associated with a numerical score for known drug discovery amenability satisfying one or more predefined threshold criteria.
85. The system of any one of embodiments 79-84, wherein receiving the training data set comprises selecting the plurality of protein candidate sites from a protein sequence data source in accordance with metadata indicating that the protein candidate sites satisfy one or more protein abundance criteria.
86. The system of any one of embodiments 79-85, wherein receiving the training data set comprises selecting the plurality of protein candidate sites from a protein sequence data source in accordance with metadata indicating that the protein candidate sites satisfy one or more isotopic ratio criteria.
87. The system of any one of embodiments 79-86, wherein receiving the training data set comprises selecting the plurality of protein candidate sites from a protein sequence data source in accordance with metadata indicating that the protein candidate sites satisfy one or more reactivity criteria.
88. The system of any one of embodiments 79-87, wherein training the classifier comprises applying a model selected from Support Vector Machines (SVM), Random Forests (RF), and eXtreme Gradient Boosting (XGBoost).
89. The system of any one of embodiments 79-88, wherein each of the feature sets of the plurality of feature sets comprises:

one or more features characterizing frequency of observation of one or more peptides associated with the respective protein candidate site across one or more experimental iterations, as indicated by metadata corresponding to the respective protein candidate site;

one or more features characterizing protein abundance for the respective protein candidate site; and

one or more features characterizing sequence characteristics associated with the respective protein candidate site.
90. A method for training a classifier for identifying protein candidate sites, the method performed at a system comprising one or more processors, the method comprising:

receiving a corpus of training data comprising data regarding a plurality of protein candidate sites;

generating, based on the training data, a plurality of feature sets corresponding to the plurality of protein candidate sites; and

training a classifier using the plurality of feature sets to classify protein candidate sites for amenability for drug-discovery.
91. A non-transitory computer-readable storage medium for training a classifier for identifying protein candidate sites, the non-transitory computer-readable storage medium storing instructions configured to be executed by a system comprising one or more processors to cause the system to:

receive a corpus of training data comprising data regarding a plurality of protein candidate sites;

generate, based on the training data, a plurality of feature sets corresponding to the plurality of protein candidate sites; and

train a classifier using the plurality of feature sets to classify protein candidate sites for amenability for drug-discovery.
92. A system for characterizing protein candidate sites, the system comprising one or more processors configured to cause the system to:

receive data comprising a set of protein candidate sites and corresponding metadata regarding experimental data associated with respective sites of the set of candidate sites;

for each protein candidate site of the set of protein candidate sites, determine, based on the received data:
a number of times that one or more peptides associated with the respective protein candidate site was observed across one or more experimental iterations; and
a number of experimental iterations in which one or more peptides associated with the respective protein candidate site was observed;

select a subset of the received data, wherein the selection is based on the number of times that the one or more peptides were observed across the one or more experimental iterations and on the number of experimental iterations in which the one or more peptides were observed, wherein the subset of the received data represents a subset of the set of protein candidate sites; and

generate and store a characterization of the subset of protein candidate sites, wherein the characterization characterizes amenability of the protein candidate sites for drug-discovery.
93. The system of embodiment 92, wherein generating the characterization comprises:

generating, for each of the respective protein candidate sites in the subset, a respective feature set; and

applying a classifier to the respective feature sets in order to generate the characterization.
94. The system of any one of embodiments 92-93, wherein the characterization of the amenability for drug-discovery of the protein candidate site comprises a probability of the protein candidate site being reactive.
95. The system of any one of embodiments 92-94, wherein the one or more processors are further configured to cause the system to generate and store a ranking of the subset of protein candidate sites, wherein the ranking is based on the characterization of the amenability for drug-discovery for one or more of the protein candidate sites.
96. The system of any one of embodiments 93-95, wherein the respective feature set comprises one or more selected from the following:

one or more features characterizing frequency of observation of one or more peptides associated with the respective protein candidate site across one or more experimental iterations, as indicated by metadata corresponding to the respective protein candidate site;

one or more features characterizing protein abundance for the respective protein candidate site; and

one or more features characterizing sequence characteristics associated with the respective protein candidate site.
97. A method for characterizing protein candidate sites, the method performed at a system comprising one or more processors, the method comprising:

receiving data comprising a set of protein candidate sites and corresponding metadata regarding experimental data associated with respective sites of the set of candidate sites;

for each protein candidate site of the set of protein candidate sites, determining, based on the received data:
a number of times that one or more peptides associated with the respective protein candidate site was observed across one or more experimental iterations; and
a number of experimental iterations in which one or more peptides associated with the respective protein candidate site was observed;

selecting a subset of the received data, wherein the selection is based on the number of times that the one or more peptides were observed across the one or more experimental iterations and on the number of experimental iterations in which the one or more peptides were observed, wherein the subset of the received data represents a subset of the set of protein candidate sites; and

generating and store a characterization of the subset of protein candidate sites, wherein the characterization characterizes amenability of the protein candidate sites for drug-discovery.
98. A non-transitory computer-readable storage medium for characterizing protein candidate sites, the non-transitory computer-readable storage medium storing instructions configured to be executed by a system comprising one or more processors to cause the system to:

receive data comprising a set of protein candidate sites and corresponding metadata regarding experimental data associated with respective sites of the set of candidate sites;

for each protein candidate site of the set of protein candidate sites, determine, based on the received data:
a number of times that one or more peptides associated with the respective protein candidate site was observed across one or more experimental iterations; and
a number of experimental iterations in which one or more peptides associated with the respective protein candidate site was observed;

select a subset of the received data, wherein the selection is based on the number of times that the one or more peptides were observed across the one or more experimental iterations and on the number of experimental iterations in which the one or more peptides were observed, wherein the subset of the received data represents a subset of the set of protein candidate sites; and

generate and store a characterization of the subset of protein candidate sites, wherein the characterization characterizes amenability of the protein candidate sites for drug-discovery.
99. A method of screening potential lead compounds against a protein, comprising:

identifying a protein having a protein candidate site characterized as amenable for drug-discovery by the method of embodiment 38, embodiment 77, or embodiment 97; and

testing one or more potential lead compounds for interaction with the protein candidate site of the protein.
100. A method of screening potential lead compounds against a protein, comprising:

identifying a protein having a protein candidate site ranked as amenable for drug-discovery by the method of embodiment 38, embodiment 77, or embodiment 97; and

testing one or more potential lead compounds for interaction with the protein candidate site of the protein.
101. The method of any one of embodiments 99-100, wherein the interaction of the one or more potential lead compounds with the protein is covalent binding of the one or more potential lead compounds with the protein.
102. The method of any one of embodiments 99-101, wherein the one or more potential lead compounds covalently bind to the protein candidate site.
103. The method of any one of embodiments 99-100, wherein the interaction of the one or more potential lead compounds with the protein is non-covalent binding of the one or more potential lead compounds with the protein.
104. The method of any one of embodiments 99-103, further comprising selecting a lead compound from the potential lead compounds.
105. The method of embodiment 104, wherein the lead compound is selected based on one or more of binding affinity to the protein candidate site, reaction kinetics with the protein candidate site, extent of covalent modification of the protein candidate site by the lead compound, amount of reaction with off-target sites in the protein, amount of reaction with off-target proteins, agonistic interaction with the protein, antagonist interaction with the protein, or selectivity for the protein candidate site.
106. The method of any one of embodiments 104-105, further comprising modifying the lead compound to enhance its binding with the protein.

Any one or more features and/or aspects of the above enumerated embodiments may be combined in whole or in part with one another and/or with any other feature and/or aspect disclosed herein.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

It is understood that the present disclosure has been made only by way of example, and that numerous changes in the combination and arrangement of parts can be resorted to by those skilled in the art without departing from the spirit and scope of present disclosure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A system for characterizing protein candidate sites, the system comprising one or more processors configured to cause the system to:
receive experimental data comprising spectral data from an experimental data source;
in response to receiving the experimental data comprising the spectral data, automatically create, based on the experimental data, a data set comprising a set of protein candidate sites within one or more proteins;
for each protein candidate site of the set of protein candidate sites, generate, based on the data set comprising the set of protein candidate sites, a feature set characterizing the respective protein candidate site; and
generate a characterization of the amenability for drug-discovery for one or more of the protein candidate sites by applying a classifier to the respective feature set for the protein candidate site.

2. A system for ingesting data from biophysical screening experiments, the system comprising one or more processors configured to cause the system to:
receive experimental data comprising spectral data from an experimental data source;
in response to receiving the experimental data comprising the spectral data, automatically generate, based on the received experimental data comprising the spectral data, for each peptide of a plurality of peptides, respective mapping data that maps a respective one of the peptides to one or more proteins of a respective plurality of proteins;
automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and
store the data set comprising the set of protein candidate sites in a first database.

3. The system of claim 2, wherein the one or more processors are further configured to cause the system to store the generated mapping data in the first database.

4. The system of claim 2, wherein generating the mapping data comprises correlating spectra from within the received spectral data to matching spectra from calculated theoretical spectra.

5. The system of claim 4, wherein the one or more processors are further configured to cause the system to calculate the theoretical spectra based on protein sequence data received by the system from a protein sequence data source.

6. The system of claim 2, wherein the one or more processors are further configured to cause the system to automatically generate and store in the first database a sequence of one or more peptides comprising the protein candidate sites.

7. The system of claim 2, wherein the one or more processors are further configured to cause the system to generate a score characterizing a confidence level associated with at least part of the data set comprising the set of protein candidate sites.

8. The system of claim 2, wherein the one or more processors are further configured to cause the system to:
detect an update to the protein sequence data source; and
in response to detecting the update to the protein sequence data source, automatically update the data set comprising the set of protein candidate sites based on updated information retrieved from the updated protein sequence data source.

9. The system of claim 8, wherein automatically updating the data set comprising the set of protein candidate sites comprises performing one or more sequence alignments for a peptide of the plurality of peptides.

10. The system of claim 9, wherein performing the one or more sequence alignments for the peptide comprises aligning the peptide against updated protein sequence information for each of the proteins to which the peptide was previously indicated, by the mapping data, as having been mapped.

11. The system of claim 8, wherein automatically updating the data set comprising the set of protein candidate sites comprises aligning each peptide of the plurality of peptides against a new protein sequence added to the protein sequence data store.

12. The system of claim 8, wherein the updated information retrieved from the updated protein sequence data store comprises one or more of: information indicating a single-residue change, information indicating an insertion of an amino acid, information indicating a deletion of an amino acid, information indicating a novel protein annotation, information indicating merging of two or more protein entries into a single protein entry, and information indicating deletion of a protein entry.

13. The system of claim 2, wherein the one or more processors are further configured to cause the system to, in response to receiving the experimental data comprising the spectral data, store the experimental data comprising the spectral data in a second database.

14. The system of claim 2, wherein the one or more processors are further configured to cause the system to:
receive metadata specifying experimental conditions for the experimental data source, wherein the metadata is received via a plurality of predefined fields for experimental condition data types; and
in response to receiving the metadata specifying the experimental conditions, generate and store a record of the experimental conditions in the first database.

15. The system of claim 2, wherein the spectral data comprises mass spectrometry data.

16. The system of claim 2, wherein the spectral data comprises tandem mass spectrometry data.

17. The system of claim 2, wherein the spectral data is received by the system before being associated with any peptides or proteins.

18. The system of claim 2, wherein the data set comprising the set of protein candidate sites comprises indication of a set of candidate residues determined by the system to be potentially modified by promiscuous probes.

19. The system of claim 2, wherein the experimental data is data generated by one or more of a cell-based screening experiment, an in vitro screening experiment, an in situ screening experiment, an in vivo screening experiment, a purified protein screening experiment, and a recombinant protein screening experiment.

20. The system of claim 2, wherein the plurality of proteins comprises one or more of a protein isoform and a protein mutant.

21. A method for ingesting data from biophysical screening experiments, the method performed at a system comprising one or more processors, the method comprising:
receiving experimental data comprising spectral data from an experimental data source;
in response to receiving the experimental data comprising the spectral data, automatically generating, based on the received experimental data comprising the spectral data, for each peptide of a plurality of peptides, respective mapping data that maps a respective one of the peptides to one or more proteins of a respective plurality of proteins;
automatically creating, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and
storing the data set comprising the set of protein candidate sites in a first database.

22. A non-transitory computer-readable storage medium for ingesting data from biophysical screening experiments, the non-transitory computer-readable storage medium storing instructions configured to be executed by a system comprising one or more processors to cause the system to:
receive experimental data comprising spectral data from an experimental data source;
in response to receiving the experimental data comprising the spectral data, automatically generate, based on the received experimental data comprising the spectral data, for each peptide of a plurality of peptides, respective mapping data that maps a respective one of the peptides to one or more proteins of a respective plurality of proteins;
automatically create, based on the one or more proteins of the respective plurality of proteins indicated by the generated mapping data, a data set comprising a set of protein candidate sites within one or more of the proteins of the respective plurality of proteins, wherein creating the data set comprising the set of protein candidate sites comprises retrieving, from a protein sequence data source, protein annotation information indicating that the protein candidate sites are associated with one or more of the proteins; and
store the data set comprising the set of protein candidate sites in a first database.
